(19) United States
(12) Patent Application Publication
(10) Pub. No.: US 2023/0257423 A1
(43) Pub. Date: Aug. 17, 2023

(54) COMPOSITIONS FOR TREATING EPITHELIAL BARRIER FUNCTION DISORDERS

(71) Applicants: ENTEROME, PARIS (FR); Institut National de recherche pour I'agriculture, I'alimentation et I'environnement, PARIS (FR)

(72) Inventors: Malgorzata NEPELSKA, VALBONNE (FR); Joel DORE, VITRY SUR SEINE (FR); Herve BLOTTIERE, NANTES (FR); Antonella CULTRONE, L'HAY LES ROSES (FR); Christophe BONNY, PARIS (FR)

(21) Appl. No.: 17/794,562

(22) PCT Filed: Jan. 22, 2021
(86) PCT No.: PCT/EP2021/051535

§ 371 Date: Jul. 21, 2022

(30) Foreign Application Priority Data
Jan. 23, 2020 (EP) 20305055.4

(51) Int. Cl. C07K 9/00; A61K 47/64

(52) U.S. Cl. CPC C07K 9/005; A61K 47/64

(57) ABSTRACT

The present disclosure relates to novel pharmaceutical compositions comprising recombinantly engineered probiotic bacteria that can be used, inter alia, in the treatment of gastrointestinal inflammatory diseases and epithelial barrier function disorders. The probiotic bacteria preferably contain a nucleic acid encoding the heterodimeric protein of SEQ ID NO:1 and 2, or homologous sequences thereof sharing at least 80% identity with said sequences. In some embodiments, the pharmaceutical compositions described herein have particular application in the treatment or prevention of disease states associated with abnormally permeable epithelial barriers as well as inflammatory bowel diseases or disorders.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Feb. 28, 2023, is named 17794562_Sequence_listing_022823.txt and is 74,333 bytes in size.

### SUMMARY OF THE INVENTION

The present disclosure relates to novel pharmaceutical compositions comprising recombinantly engineered probiotic bacteria that can be used, inter alia, in the treatment of gastrointestinal inflammatory diseases and epithelial barrier function disorders. In some embodiments, the pharmaceutical compositions described herein have particular application in the treatment or prevention of disease states associated with abnormally permeable epithelial barriers as well as inflammatory bowel diseases or disorders.

### BACKGROUND OF THE INVENTION

Numerous diseases and disorders are associated with decreased gastrointestinal epithelial cell barrier function or integrity. One such disease is inflammatory bowel disease (IBD), the incidence and prevalence of which is increasing with time and in different regions around the world, indicating its emergence as a global disease. “IBD” is a collective term that describes conditions with chronic or recurring immune response and inflammation of the gastrointestinal (GI) tract. The two most common inflammatory bowel diseases are ulcerative colitis (UC) and Crohn's disease (CD).

Crohn's disease (CD) is a chronic inflammatory bowel disease (IBD) that may affect any part of the gastrointestinal tract from mouth to anus. The age of onset is generally between 15-30 years and it is equally prevalent in women and men. The highest prevalence is found in Europe and North America with just over 300 per 100.000 persons (Molodecky et al., Gastroenterology. 2012 January; 142(1):46-54). CD generally leads to abdominal pain, severe diarrhea and weight disorders. The disease is of unknown etiology and multifactorial: environmental factors, host genetics and gut microbiome have all been shown to impact the risk of disease and its severity (Cho et al., Gastroenterology, 2011 May; 140(6), 1704-12). The clinical diagnosis of CD is supported by serologic, radiologic, endoscopic, and histologic findings.

Ulcerative colitis (or UC) is another form of inflammatory bowel disease (IBD). Ulcerative colitis is a form of colitis, a disease of the colon (the largest portion of the large intestine), that includes characteristic ulcers, or open sores. The main symptom of active disease is usually constant diarrhea mixed with blood, of gradual onset.

Disease progression relies on a breakdown of intestinal barrier function that allows bacteria or bacterial components to translocate into mucosal tissue (Maloy K. et al, 2011. Nature, June 15; 474 (7531) 298-306; Martini et al., 2017, Cell Mol Gastroenterol Hepatol, 4:33-46). Bacterial translocation results in activation of inflammatory signalling which triggers additional barrier disruption, resulting in a cyclic amplification loop of barrier disruption, bacterial translocation and inflammation. While many current therapies target inflammation, the lack of therapies promoting mucosal healing provides an opportunity for novel therapies promoting epithelial repair and intestinal barrier integrity.

Currently, many IBD therapeutics available in the market merely aim to target and suppress the inflammatory response associated with IBD. While helpful, this narrow therapeutic mode of action disregards the important contribution that epithelial barrier integrity plays in the aetiology of the disease.

Thus, there is a great need in the art for the development of therapeutic tools, which not only suppress the immune system's inflammatory response, but that also act in concert to restore the epithelial barrier function in an individual.

### DESCRIPTION OF THE INVENTION

While uses of live microbial populations to treat diseases is increasingly common, such methods rely on the ability of the administered bacteria to survive in the host or patient and to interact with the host tissues in a beneficial and therapeutic way. An alternative approach, provided here, is to identify microbially-encoded proteins and variants thereof which can affect cellular functions in the host and provide therapeutic benefit. Such benefit can be obtained, for example, by administering live biotherapeutic bacteria that have been engineered to express a therapeutic protein or help secrete a therapeutic protein. Interestingly, methods of treatment comprising administration of the bacteria are not limited to the gut (small intestine, large intestine, rectum) but may also include treatment of other disorders within the alimentary canal (such as oral mucositis).

Starting from a metagenomic clone identified through high throughput screening on a NE-KB reporter system, the present inventors were able to identify a bacterial clone of interest that present an anti-inflammatory activity. More precisely, they herein show that this clone of interest is able to secrete bioactive compounds that protect the epithelial barrier from bacterial infection. During their investigations, the present inventors identified a new sequence of interest that encodes an heterodimeric transporter (MsbA1-MsbA2-like), and that is located on the bacterial membrane. Because of the anti-inflammatory properties of the supernatant of the selected clone, it was speculated that this transporter is able to help the secretion of bioactive anti-inflammatory molecules into the supernatant. Three compounds have been identified, that are specifically present in the anti-inflammatory supernatant of this clone and can be responsible of the anti-inflammatory biological activity of this supernatant. All three are muropeptide precursors. Although these compounds are normally found only inside Gram negative bacteria, it is now speculated that the heterodimeric transporter can trigger the turnover/transport of these compounds outside bacterial cells, thereby enhancing the secretion of anti-inflammatory compounds such as muropeptide precursors.

The purification of the fractions secreted by the selected clones resulted in the identification of the three following muropeptide precursors:

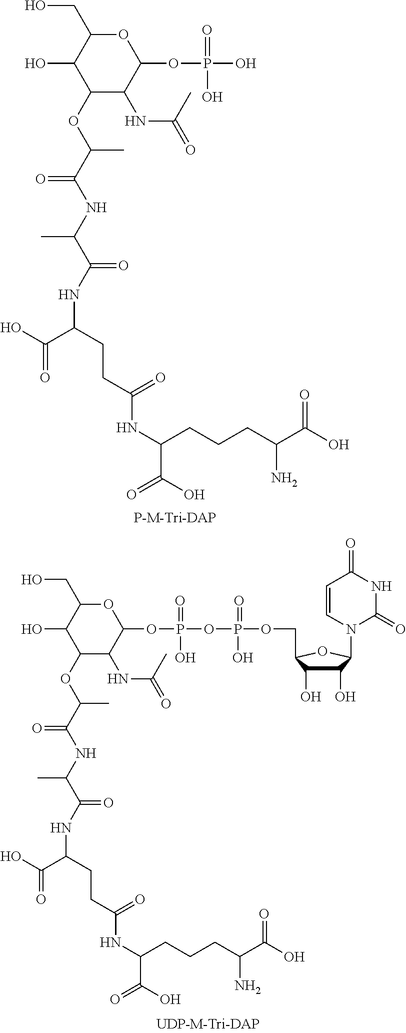

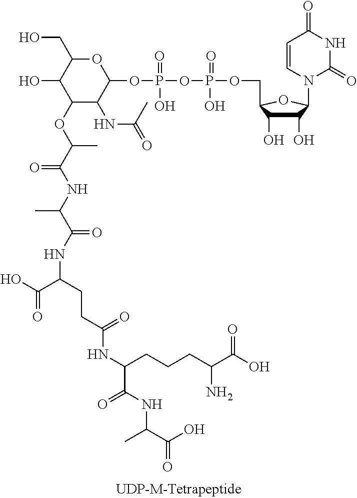

The present invention relies on the discovery of a new sequence of interest, with which it is possible to enhance the secretion of these anti-inflammatory compounds in bacteria. Notably, it relates to the nucleotide sequence per se, vectors for expressing same, and bacteria cells that have been genetically modified so as to express said transporter at their membrane. As shown below in the experimental part, the recombinant bacteria can secrete bioactive anti-inflammatory compounds that protect the epithelial barrier from bacterial infection and associated inflammation. These genetically engineered bacteria, which will therefore be useful for treating disorders, are associated with decreased gastrointestinal epithelial cell barrier function or integrity, such as IBDs. The present invention also relates to therapeutic compositions containing these anti-inflammatory compounds.

Nucleic Acid Molecules of the Invention

In a first aspect, the present invention relates to an isolated or recombinant nucleic acid encoding a heterodimeric protein comprising a first polypeptide whose amino acid sequence is SEQ ID NO: 1 or a homologous sequence thereof, and a second polypeptide whose amino acid sequence is SEQ ID NO: 2 or a homologous sequence thereof.

In a preferred embodiment, said isolated or recombinant nucleic acid encodes a heterodimeric protein whose first and second polypeptides have a sequence of at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with SEQ ID NO:1 and SEQ ID NO:2 respectively, said encoded heterodimeric protein having the same biological function as the heterodimeric protein of SEQ ID NO:1 & 2.

The biological function of the heterodimer of SEQ ID NO:1 & 2 is linked to its beneficial effects on the epithelial barrier function, as measured for example on ileal explants treated with live bacteria (see the in vitro TEER assays disclosed in the examples: TEER assays are well-known methods for measuring effects on the structural and functional integrity of an epithelial cell layer (Srinivasan et ai., 2015, j Lab Autom, 20: 107-126)) or on the upregulation of various genes linked to tight junctions in epithelial cells. Preferably, this function is assessed by evaluating the secretion of the anti-inflammatory cytokine IL-10 by Monocyte-derived Dendritic Cells (hereafter called DCs) by conventional means, for example with research tools such as the AlphaLISA research reagents for IL10 measurement, product AL218 C/F sold by PerkinElmer. It is also possible to assess that the biological function of SEQ ID NO:1 &2 is fulfilled by measuring the amount of muropeptide precursors that are secreted in the extracellular medium, for example by mass spectrometry, as disclosed in the experimental part below (see FIG. 7). The biological function of the transporter of the invention is fulfilled if the host cell, e.g., a non-pathogenic Gram-negative bacterium, is able to secrete at least 1 μM, preferably at least 2 μM, more preferably at least 5 μM of at least one muropeptide precursor, such as those defined in the invention (M-Tri-DAP-MP, UDP-M-tri-DAP, or UDP-M-Tetra-DAP). Preferably, the polypeptide of the invention is functional in the transformed host cell, e.g., a non-pathogenic Gram-negative bacterium, if said host cell is able to secrete at least 1 μM, preferably at least 2 μM, more preferably at least 5 μM of the three muropeptide precursors of the invention (M-Tri-DAP-MP, UDP-M-tri-DAP, or UDP-M-Tetra-DAP).

In a more preferred embodiment, said isolated or recombinant nucleic acid encodes a heterodimeric protein whose first and second polypeptides have the sequences SEQ ID NO:1 and SEQ ID NO:2 respectively.

In a particular aspect, said recombinant nucleic acid comprises, in the same Open Reading Frame (ORF), a first polynucleotide having the sequence SEQ ID NO:3 and a second polynucleotide having the sequence SEQ ID NO:4, or homologous sequences thereof. In a preferred embodiment, said recombinant nucleic acid comprises, in the same ORF, a first polynucleotide having the sequence SEQ ID NO:3 and a second polynucleotide having the sequence SEQ ID NO:4, as disclosed in the enclosed listing. More preferably, they are consecutively located, as depicted in SEQ ID NO:8.

By “recombinant”, it is herein meant that the nucleic acid sequence of the invention is not necessarily found in nature. It refers for example to a molecule comprising nucleic acid sequences that are joined together or produced by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention. The recombinant nucleic acids of the invention will be introduced into a host cell in order to express the appropriate transporter heterodimeric polypeptide.

Importantly, the recombinant nucleic acids of the invention are able to express or to encode the heterodimeric polypeptide of the invention once they are introduced in host cells, meaning that they contain the necessary information for the host cell machinery to express the resulting polypeptide in a sufficient amount. Thus, the recombinant nucleic acids of the invention do not only “comprise” SEQ ID NO:3 or 4 or homologous thereof, but also have them in the same operon or ORF or in appropriate conditions for them to be efficiently encoded by a host machinery.

Such recombinant nucleic acids may include sequences derived from the same host cell species or from different host cell species, which have been isolated and reintroduced into cells of the host species.

Recombinant nucleic acids sequences may remain free (i.e., non-integrated into a host cell genome), or may become integrated (“stably” or “temporary” incorporated) into the host cell genome, either as a result of the original transformation of the host cells, or as the result of subsequent recombination and/or repair events. Still, they remain “heterologous” as compared to the host cell and its natural genome.

By “heterologous” is herein meant a nucleic acid (or polypeptide molecule) that has been manipulated by human intervention so that it is located in a place other than the place in which it is naturally found. For example, a nucleic acid sequence from one species may be introduced into the genome of another species, or a nucleic acid sequence from one genomic locus may be moved to another genomic locus in the same species. A heterologous protein includes, for example, a protein expressed from a heterologous coding sequence or a protein expressed from a recombinant gene in a cell that would not naturally express the protein.

In a further particular aspect, the recombinant nucleic acid of the invention comprises the polynucleotide having the sequence SEQ ID NO:5, as depicted in the enclosed listing. This sequence contains SEQ ID NO:3, SEQ ID NO:4 and naturally occurring regulatory sequences that may have an influence on the expression of the heterodimeric protein of the invention in the Firmicutes Gram-positive bacterium from which the sequences have been cloned. In this embodiment, the recombinant nucleic acid of the invention may comprise, apart from SEQ ID NO:5, any other nucleic acid sequence that is not naturally associated with SEQ ID NO:5.

In another particular aspect, said first and second recombinant polynucleotides are operably linked to regulatory sequences allowing their expression in other host cells, or to other regulatory sequences allowing their expression in a host cell, preferably in a bacterial host cell, e.g., a Escherichia coli Gram-negative bacterium. These regulatory sequences are well-known in the art. They are for example a promoter. Prokaryotic promoters typically fall into two classes, inducible and constitutive. An inducible promoter is a promoter that initiates increased levels of transcription of the encoding polynucleotide under its control in response to changes in the culture condition, e.g., the presence or absence of a nutrient or a change in temperature. A large number of promoters recognized by a variety of potential host cells are well known and a skilled artisan can choose the promoter according to desired expression levels. Promoters suitable for use with prokaryotic hosts include E. coli promoters such as lac, trp, tac, trc and ara, viral promoters recognized by E. coli such as lambda and T5 promoters, and the T7 and TTlac promoters derived from T7 bacteriophage. In preferred embodiments, the promoter is an inducible promoter which is under the control of chemical or environmental factors.

In a preferred embodiment, the recombinant nucleotide of the invention contains promoter that is not naturally associated with SEQ ID NO:3 and SEQ ID NO:4. More precisely, the recombinant nucleotide of the invention preferably does not contain the natural promoter associated with SEQ ID NO:3 and SEQ ID NO:4 in a Firmicutes Gram-positive bacterium. It can however contain any other promoter that is efficient in a host cell, and more precisely in bacteria.

The present invention relates, of course, to both the DNA and RNA sequences, and also the sequences which hybridize with them, as well as the corresponding double-stranded DNAs.

As used herein, the terms “nucleic acid”, “nucleic acid sequence” or “sequence of nucleic acid”, “polynucleotide”, “oligonucleotide”, “polynucleotide sequence”, and “nucleotide sequence”, which will be used equally in the present description, will be intended to refer to double-stranded DNA, single-stranded DNA and products of transcription of said DNAs. This nucleic acid molecule may include non-natural nucleotides.

In the present specification, the “non-natural nucleotide” refers to an artificially constructed or artificially chemically modified nucleotide and refers to a non-naturally occurring nucleotide similar in properties and/or structure to the natural nucleotide, or a non-naturally occurring nucleotide comprising a nucleoside or a base similar in properties and/or structure to a nucleoside or a base constituting the natural nucleotide. Examples thereof include a basic nucleoside, arabinonucleoside, 2′-deoxyuridine, α-deoxyribonucleoside, β-L-deoxyribonucleoside, and other glycosylated nucleosides. The glycosylated nucleosides include glycosylated nucleosides having substituted pentose (2′-O-methyl ribose, 2′-deoxy-2′-fluororibose, 3′-O-methylribose, or 1′,2′-deoxyribose), arabinose, substituted arabinose sugar, substituted hexose, or an alpha anomer. The non-natural nucleotide present in the molecules of the present invention may be an artificially constructed base analog or an artificially chemically modified base (modified base). Examples of the “base analog” include a 2-oxo(1H)-pyridin-3-yl group, a 5-substituted 2-oxo(1H)-pyridin-3-yl group, a 2-amino-6-(2-thiazolyl)purin-9-yl group, a 2-amino-6-(2-thiazolyl)purin-9-yl group, and a 2-amino-6-(2-oxazolyl)purin-9-yl group. Examples of the “modified base” include modified pyrimidine (e.g., 5-hydroxycytosine, 5-fluorouracil, and 4-thiouracil), modified purine (e.g., 6-methyladenine and 6-thioguanosine), and other heterocyclic bases.

More specifically, the non-natural nucleotide contained in the nucleic acid system of the invention is an artificially constructed nucleic acid analog similar in structure and/or properties to the natural nucleic acid. Examples thereof include a peptide nucleic acid (PNA), a peptide nucleic acid having a phosphate group (PHONA), a bridged nucleic acid or locked nucleic acid (BNA or LNA), and a morpholino nucleic acid. The non-natural nucleotide can also include chemically modified nucleic acids or nucleic acid analogs such as methylphosphonate-type DNA or RNA, a phosphorothioate-type DNA or RNA, phosphoramidate-type DNA or RNA, and 2′-O-methyl-type DNA or RNA.

It should be understood that the present invention does not relate to the genomic nucleotide sequences in their natural chromosomal environment, i.e., in their natural state. It involves sequences which have been “isolated” and/or “purified”, i.e., they have been removed, directly or indirectly, from their natural chromosomal environment, for example by copying, synthetizing, etc.

In the context of the nucleic acids of the invention, the term “homologous sequence” is generally intended to refer to a sequence which has, with respect to the reference nucleic acid sequence, certain modifications, such as in particular a deletion, a truncation, an extension, a chimeric fusion and/or a mutation, in particular a point mutation. In such cases, the homologous nucleic acid sequence can show at least 80%, preferably 90% or 95%, identity with the reference nucleic acid sequence. In the context of the nucleic acids of the invention, the term “homologous sequence” can also refer to completely different nucleic acids sequences that, due to codon degeneration, encode the same polypeptides of the invention. Codon optimization is discussed below. In any cases, the function of the said nucleic acids is the same as the function of the reference nucleotides, and that is to encode a functional polypeptide sequence that is part of the heterodimeric protein of the invention.

For the purpose of the present invention, the term “percentage of identity” between two nucleic acid or amino acid sequences is intended to refer to a percentage of nucleotides or of amino acid residues which are identical between the two sequences to be compared, obtained after the best alignment, this percentage being purely statistical and the differences between the two sequences being distributed randomly and throughout their length. Sequence comparisons between two nucleic acid or amino acid sequences are traditionally carried out by comparing these sequences after having optimally aligned them, said comparison being carried out by segment or by “window of comparison” in order to identify and compare local regions of sequence similarity. The optimal alignment of the sequences for comparison can be produced, besides manually, by means of the global homology algorithm of Needleman and Wunsch (1970) [J. Mol. Biol. 48:443]. The percentage of identity is calculated by determining the number of identical positions for which the nucleotide or the amino acid residue is identical between the two sequences, dividing this number of identical positions by the total number of positions and multiplying the result obtained by 100 so as to obtain the percentage of identity between these two sequences. For example, the needle program available on the site ebi.ac.uk, may be used, the parameters used being those given by default (in particular for the parameters “Gap open”: 10, and “gap extend”: 0.5; the matrix chosen being, for example, the “BLOSUM 62” matrix proposed by the program), the percentage of identity between the two sequences to be compared being calculated directly by the program.

The present invention also relates to nucleic acid molecules such as primers or probes which hybridize specifically with the nucleic acid molecule of sequence SEQ ID NO:3 and/or SEQ ID NO:4. These nucleic acid molecules have preferably at least 80%, preferably at least 90% or at least 95% identity with a fragment of the complementary sequence of SEQ ID NO:3 and/or SEQ ID NO:4. Specific hybridization is preferably observed under high stringency conditions, i.e., when the temperature and ionic strength conditions are chosen so as to allow the hybridization between two complementary DNA fragments to be maintained. By way of illustration, high stringency conditions can be as follows. The DNA-DNA or DNA-RNA hybridization is carried out in two steps: (1) prehybridization at 42° C. for 3 hours in phosphate buffer (20 mM, pH 7.5) containing 5*SSC (1*SSC corresponds to a 0.15 M NaCl+0.015 M sodium citrate solution), 50% of formamide, 7% of sodium dodecyl sulfate (SDS), 10*Denhardt's, 5% of dextran sulfate and 1% of salmon sperm DNA; (2) actual hybridization for 20 hours at a temperature dependent on the size of the probe (i.e. 42° C. for a probe of size>100 nucleotides), followed by two 20-minute washes at 20° C. in 2*SSC+2% SDS and one 20-minute wash at 20° C. in 0.1*SSC+0.1% SDS. The final wash is carried out in 0.1*SSC+0.1% SDS for 30 minutes at 60° C. for a probe of size>100 nucleotides. The high stringency hybridization conditions described above for a polynucleotide of defined size will be adjusted by those skilled in the art for oligonucleotides of greater or smaller size, according to the teaching of Sambrook et al., 1989.

Alternatively, the present invention relates to a nucleic acid molecule encoding fragments of the polypeptides of SEQ ID NO:1 and/or SEQ ID NO:2, or of a homolog thereof. Said polypeptide fragments are characterized in that they comprise for example at least 300 consecutive amino acids, preferably at least 400 or at least 500 consecutive amino acids of SEQ ID NO:1 and/or SEQ ID NO:2, or of a homolog thereof.

The present invention also targets an isolated or recombinant nucleic acid molecule characterized in that it encodes at least 300 consecutive amino acids, preferably at least 350, at least 400, at least 450, at least 500, at least 525, at least 550, at least 575 amino acids of a sequence chosen from the group comprising:

a) the sequence SEQ ID NO:1 and/or SEQ ID NO:2,

b) an homologous sequence of SEQ ID NO:1 and/or SEQ ID NO:2.

Said polypeptide fragment has preferably the same biological function as the heterodimeric protein of SEQ ID NO:1 & 2.

This function is linked to its beneficial effects on the epithelial barrier function, as measured for example on ileal explants treated with live bacteria (see the in vitro TEER assays disclosed in the examples: TEER assays are well-known methods for measuring effects on the structural and functional integrity of an epithelial cell layer (Srinivasan et ai., 2015, j Lab Autom, 20: 107-126)) or on the upregulation of various genes linked to tight junctions in epithelial cells.

Preferably, this function is assessed by evaluating the secretion of the anti-inflammatory cytokine IL-10 by Monocyte-derived Dendritic Cells (hereafter called DCs) by conventional means, for example with research tools such as the AlphaLISA research reagents for IL10 measurement, product AL218 C/F sold by PerkinElmer.

It is also possible to assess this function by measuring the amount of muropeptide precursors that are secreted in the extracellular medium, for example by mass spectrometry, as disclosed in the experimental part below (see FIG. 7). The biological function of the transporter of the invention is fulfilled if the host cell, e.g., a non-pathogenic Gram-negative bacterium, is able to secrete at least 1 μM, preferably at least 2 μM, more preferably at least 5 μM of at least one muropeptide precursor, such as those defined in the invention (M-Tri-DAP-MP, UDP-M-tri-DAP, or UDP-M-Tetra-DAP). Preferably, the polypeptide of the invention is functional in the transformed host cell, e.g., a non-pathogenic Gram-negative bacterium, if said host cell is able to secrete at least 1 μM, preferably at least 2 μM, more preferably at least 5 μM of the three muropeptide precursors of the invention (M-Tri-DAP-MP, UDP-M-tri-DAP, or UDP-M-Tetra-DAP).

More generally, the present invention targets an isolated or recombinant nucleic acid molecule characterized in that it comprises at least 900 consecutive nucleotides, preferably at least 1000, at least 1100, at least 1200, at least 1300, at least 1400, at least 1500, at least 1600, at least 1700 or at least 1750 consecutive nucleotides of a sequence chosen from the group comprising:

a) the sequence SEQ ID NO:3 and/or SEQ ID NO:4,

b) an homologous sequence of SEQ ID NO:3 and/or SEQ ID NO:4,

c) the sequence of a variant of the SEQ ID NO:3 and/or SEQ ID NO:4,

d) a sequence which is complementary to the nucleic acid of sequence SEQ ID NO:3 and/or SEQ ID NO:4; and

e) the sequence of the corresponding RNAs.

The term “variant” is hereby intended to refer to a nucleic acid whose sequence contains individual variations as compared with the reference nucleic acid sequence SEQ ID NO:3 and/or SEQ ID NO:4. These natural mutated sequences correspond to polymorphisms present in bacteria.

In a preferred embodiment, the present invention relates to fragments of the nucleic acid molecule of SEQ ID NO:3 and/or SEQ ID NO:4, a homolog or a variant thereof. Said fragments are characterized in that they comprise at least 900 consecutive nucleotides, preferably at least 1500 or at least 1700 consecutive nucleotides of SEQ ID NO:3 and/or SEQ ID NO:4, a homolog or a variant thereof, and they encode the polypeptide of the invention of SEQ ID NO:1 and/or SEQ ID NO:2, or a homologous or fragment thereof, having the same biological function.

In some embodiments, the polynucleotide encoding the heterodimeric protein of the invention, or the fragment or sequence variant thereof, may be codon-optimized. The skilled artisan is aware of various tools for codon optimization, such as those described in: Ju Xin Chin, et al, Bioinformatics, Volume 30, Issue 15, 1 Aug. 2014, Pages 2210-2212; or in: Grote A, et al, Nucleic Acids Res. 2005 Jul. 1; 33(Web Server issue:W526-31; or in US 2011/0081708 A1; or as provided by commercial suppliers, e.g., the codon optimization algorithm provided by Twist Bioscience (San Francisco, USA). In some embodiments, the polynucleotide encoding the heterodimeric protein of the invention, or the fragment or sequence variant thereof, is codon-optimized for expression by prokaryotic cells, preferably it is codon-optimized for expression in bacteria, such as E. coli. In some embodiments, the polynucleotide encoding the heterodimeric protein of the invention contains at least one codon which is not present in the natural sequence SEQ ID NO:3 or SEQ ID NO:4, said modified codon encoding the same amino acid as the natural codon.

Polypeptides of the Invention

In another aspect, the present invention relates to the recombinant polypeptides encoded by the isolated polynucleotides described above.

The term “recombinant” when made in reference to a protein or a polypeptide refers to a protein or polypeptide molecule that is expressed using a recombinant nucleic acid construct created by means of molecular biological techniques.

In particular, the present invention relates to a heterodimeric protein comprising a first polypeptide whose amino acid sequence is SEQ ID NO: 1, and a second polypeptide whose amino acid sequence is SEQ ID NO: 2, or homologous sequences thereof.

The term “homologous sequence” is herein intended to refer to a sequence which has, with respect to the amino acid sequences SEQ ID NO:1 or 2, certain modifications, such as in particular a deletion, a truncation, an extension, a chimeric fusion and/or a mutation, in particular a point mutation. Said homologous sequence typically shows at least 80%, preferably 90%, 91%, 92%, 93%, 94%, 95%, 96%, 97%, or at least 98% or 99% identity with the reference amino acid sequences SEQ ID NO:1 or 2, and has the same biological function as the reference polypeptides do.

In a preferred embodiment, the sequence of said heterodimeric protein has at least 80%, more preferably at least 85%, 90%, 91%, 92%, 93%, 94%, 95%, 96%, 97%, or at least 98% or 99% identity with SEQ ID NO:9, and has the same biological function as SEQ ID NO:9 does. Said heterodimeric protein has for example the amino acid sequence SEQ ID NO:9.

The present invention also relates to fragments of said polypeptides of SEQ ID NO:1 or 2 or 9, or of homologous thereof, said fragment containing at least 8, at least 10, at least 15, and more preferably at least 20 consecutive amino acids thereof. Said fragments can be used for example for generating antibodies directed against the heterodimeric protein of the invention.

In the present description, the term “polypeptide” will be used to refer equally to a “protein” or a “peptide”.

In particular, the present invention relates to a polypeptide whose amino acid sequence comprises or consists of a sequence chosen from the following group:

a) the SEQ ID NO:1 (corresponding to the amino acid sequence encoded by SEQ ID NO:3),

b) the sequence of a homolog or variant polypeptide of SEQ ID NO:1, having the same function as SEQ ID NO:1 does,

c) the sequence of a fragment thereof having the same function as SEQ ID NO:1 does.

Also, the present invention relates to a polypeptide whose amino acid sequence comprises or consists of a sequence chosen from the following group:

a) the SEQ ID NO:2 (corresponding to the amino acid sequence encoded by SEQ ID NO:4),

b) the sequence of a homolog or variant polypeptide of SEQ ID NO:2, having the same function as SEQ ID NO:2 does,

c) the sequence of a fragment thereof having the same function as SEQ ID NO:2 does.

Finally, the present invention relates to a polypeptide whose amino acid sequence comprises or consists of a sequence chosen from the following group:

a) the SEQ ID NO:9 (corresponding to the amino acid sequence encoded by SEQ ID NO:8, where SEQ ID NO:3 and SEQ ID NO:4 are consecutive),

b) the sequence of a homolog or variant polypeptide of SEQ ID NO:9, having the same function as SEQ ID NO:9 does,

c) the sequence of a fragment thereof having the same function as SEQ ID NO:9 does.

Said polypeptide “fragments” are characterized in that they comprise for example at least 300 consecutive amino acids, preferably at least 400 or at least 500 consecutive amino acids of SEQ ID NO:1 and/or SEQ ID NO:2 and/or SEQ ID NO:9, or of a homolog thereof.

The term “variant polypeptide” (or protein variant) herein designates a polypeptide which is encoded by the variant nucleic acid sequences as defined above.

It should be understood that the invention does not relate to polypeptides in natural form, i.e., they are not taken in their natural environment. Specifically, the invention relates to the peptides which are obtained by genetic recombination or by chemical synthesis. The production of a recombinant polypeptide, which can be carried out using one of the nucleotide sequences according to the invention, is particularly advantageous since it makes it possible to obtain an increased degree of purity of the desired polypeptide.

The present invention also relates to methods for producing the MsbA1-MsbA2-like transporter polypeptide of the invention, said methods comprising:
propagating a host cell comprising a recombinant nucleic acid encoding a MsbA1-MsbA2-like transporter polypeptide under conditions that allow expression of the MsbA1-MsbA2-like transporter polypeptide; and
expressing the MsbA1-MsbA2-like transporter polypeptide in the host cells,

wherein the expression of the MsbA1-MsbA2-like transporter polypeptide enhances the secretion of muropeptide precursors from the host cells.

Other methods for producing a MsbA1-MsbA2-like transporter polypeptide can comprise the following steps:
introducing a recombinant nucleic acid encoding MsbA1-MsbA2-like transporter polypeptide, as defined above, into a host cell;
selecting host cell containing the recombinant nucleic acid encoding MsbA1-MsbA2-like transporter polypeptide; and
expressing the MsbA1-MsbA2-like transporter polypeptide.

All these steps can be performed by using conventional biological tools.

All the features (host cells, nucleic acid sequences, polypeptide sequences, etc.) are as defined above and below.

TABLE 1
summary of the sequences of interest

| SEQ ID | Target | |
|---|---|---|
| NO: | sequence | Sequence |
| 1 | Polypeptide | MFQLKWVWKQMEGFRKRYIFALFSTALLAMLTLGNSVITASIMDTVFQ |
| | MsbA1-like | PLTESGVVTQQVVHHLAVLVAVLIGFTLFRTSFQYLSIMTYEGCSQKL |
| | | IFKLRRDLYKNMQEQDQDFFSKTRTGDLMTRLTGDLDMVRFAVAWVVR |
| | | QLIHCTVLFVTTSIVFLVTDWLFALSMLAVTPIIFALTLAFSKRVHPL |
| | | YVDLRERLSLLNTQAQENISGNRVVKAFAREDYEIDRFDEKNADYKKA |
| | | NTRASLLWLQYSPYIEGLSQSLSIAVLLVGGVFLITGRISIGTFTLFN |
| | | GLTWTLTDPMRMLGMHLNDLQRFFASSNKIIELYYAKSTITSRPDAKK |
| | | VDTRLKGEIDFSGVDLNLHGQPVLRHIDLHINPGETVAIMGPTGSGKT |
| | | SLVNLIPRFTDVSGGTLTIDGTPVGRYDLQGLRHAIGIATQDVFLFSD |
| | | TVDGNIAYGDSSLSEDDVKRYAKMADVDFVEKLPDGFDTLIGERGTGL |
| | | SGGQKQRIALARALAVRPSILILDDTTSAVDLETEKYIQEQLASLDFP |
| | | CTKIIVAQRISTTKRADKIVILDKGRVVDIGTHEELSQRPGYYREVFL |
| | | LQNGMEEEKEVV |
| 2 | Polypeptide | MARNKFDVDETLETPFNIKHLLRAGVYIGRHKKKMILSLLFSAISAAC |
| | MsbA2-like | SLLGPMLIQRAIDVSVPQKDYTELVVLAVIMLVSIVASVLFARARSKY |
| | | MIVVGQEIIYDIRKDLFEHLQKLPFQFYDDRPHGKILTRVINYVNSVS |
| | | DALSNGIINFVLEIFNLILIAVFMFLCDVRLSLIVMAGIPLFLVIVLL |
| | | IKPAQRRAWQDVSNKSSNINAYLHESLDGMKITQAFTREEENRGIYEK |
| | | LNKKCYQTWMKAQYTSNLVWYSVDNISTWVVGAMYLIGLWMLGPAMQI |
| | | GTLIAISSYAWRFWQPILNLSNLYNTFINAVAYLERIFEMIDEPVTVD |
| | | DAPGATELPPITGRVTFDDVTFSYDGQINILEHFNLDVKPGESIALVG |
| | | PTGAGKTTVVNLISRFYNIDRGRLLLDGHDIAQVTLRSLRSQMGIMLQ |
| | | DSFIFSGTIMDNIRYGRLDATDEEVIAAAKTVRADEFIREMEDGYYTQ |
| | | VNERGSRLSQGQRQLVAFARTLLSDPKILVLDEATSSIDAKTERLVQE |
| | | GLNALLKGRTSFIIAHRLSTIKNCDRILYISNKGIAEMGTHQQLLEKK |
| | | GYYYHLYTAQLES |
| 3 | Nucleotide | ATGTTTCAGTTAAAGTGGGTGTGGAAGCAGATGGAGGGCTTCCGAAAG |
| | encoding | CGGTACATCTTTGCCCTGTTCTCCACGGCCCTGCTGGCGATGCTGACC |
| | MsbA1-like | CTGGGCAACTCAGTCATCACCGCCAGCATTATGGACACGGTGTTCCAG |
| | | CCGCTTACCGAAAGCGGTGTGGTCACCCAGCAGGTGGTGCACCATCTG |
| | | GCGGTTCTGGTTGCGGTGCTCATTGGGTTTACGTTGTTCCGCACCAGC |
| | | TTTCAGTACCTATCCATTATGACCTATGAGGGCTGTTCCCAAAAGCTA |
| | | ATCTTCAAGCTCCGCCGGGATTTGTACAAGAATATGCAGGAGCAGGAC |
| | | CAGGACTTCTTCTCCAAAACCCGCACCGGCGACCTGATGACCCGCCTC |
| | | ACCGGCGACCTGGATATGGTCCGGTTTGCCGTGGCCTGGGTGGTGCGG |
| | | CAGCTGATCCACTGCACAGTGCTGTTTGTCACCACCTCCATTGTGTTT |
| | | TTGGTGACAGACTGGCTCTTTGCCCTGTCCATGCTGGCGGTAACGCCC |
| | | ATTATCTTTGCCCTGACGCTGGCCTTCTCCAAGCGGGTGCACCCCCTG |
| | | TATGTGGACCTGCGGGAGCGCCTCTCTTTGCTAAACACCCAGGCCCAG |
| | | GAGAACATCTCTGGCAACCGGGTGGTAAAGGCCTTCGCCCGGGAGGAC |
| | | TATGAGATCGACCGGTTCGATGAGAAAAATGCCGACTATAAAAAGGCC |
| | | AACACCAGGGCCTCCCTGCTGTGGCTGCAGTACAGCCCCTATATTGAG |
| | | GGCCTGTCCCAGTCCCTCTCCATTGCGGTGCTGCTGGTGGGGGGCGTG |
| | | TTCCTCATCACCGGGCGCATCTCAATTGGGACCTTCACCCTGTTCAAC |
| | | GGCTTGACCTGGACGCTGACCGACCCCATGCGCATGTTGGGCATGCAC |
| | | CTCAACGACCTGCAGCGTTTCTTCGCCAGTTCCAATAAGATTATCGAG |
| | | CTGTACTATGCCAAGTCCACCATCACTTCCCGGCCCGACGCCAAAAAG |
| | | GTGGACACCCGCCTGAAGGGGGAGATCGACTTCTCCGGCGTGGACCTG |
| | | AACCTCCACGGCCAACCGGTGCTGCGCCACATTGACCTGCATATCAAT |
| | | CCTGGCGAAACGGTGGCCATTATGGGGCCCACCGGCTCTGGAAAGACC |
| | | TCGCTGGTGAATTTGATCCCCCGGTTTACGGACGTCAGCGGCGGCACC |
| | | CTCACCATAGACGGCACGCCGGTGGGGCGCTATGACCTGCAGGGCCTG |
| | | CGCCACGCTATCGGTATCGCCACCCAGGACGTGTTCCTGTTTTCCGAC |
| | | ACGGTGGACGGCAATATCGCCTATGGCGACTCCTCCCTCTCCGAGGAT |
| | | GACGTGAAGCGCTATGCCAAAATGGCGGATGTGGACTTTGTGGAAAAG |
| | | CTTCCCGATGGCTTCGACACCCTCATTGGCGAGCGGGGCACCGGCCTT |
| | | TCCGGCGGCCAGAAGCAGCGCATCGCCCTGGCCCGGGCCCTGGCCGTG |
| | | CGGCCCTCCATCCTCATCTTGGATGACACCACCAGCGCCGTGGACCTG |
| | | GAAACCGAGAAGTACATCCAAGAGCAGCTGGCCAGCTTGGACTTCCCC |
| | | TGCACCAAGATCATCGTGGCCCAGCGCATTTCCACCACCAAGCGGGCG |
| | | GATAAGATCGTCATTTTGGATAAGGGCCGGGTAGTGGACATCGGCACC |
| | | CACGAGGAGCTGTCCCAGCGGCCCGGCTACTACCGGGAAGTGTTCCTG |
| | | CTGCAAAACGGCATGGAAGAGGAAAAGGAGGTGGTTTAA |
| 4 | Nucleotide | ATGGCACGCAACAAGTTTGACGTGGACGAAACCCTGGAAACCCCATTT |
| | encoding | AACATAAAGCATCTGCTCCGGGCCGGCGTGTACATTGGCCGCCACAAG |
| | MsbA2-like | AAAAAGATGATTTTGTCCCTGCTGTTCTCCGCCATTTCCGCCGCCTGC |
| | | TCTCTGCTGGGGCCCATGCTCATCCAGCGGGCCATTGACGTGTCCGTG |
| | | CCCCAAAAGGACTACACCGAGCTGGTGGTGCTGGCCGTCATCATGCTG |
| | | GTGTCCATTGTGGCCTCTGTGCTGTTCGCCCGGGCCCGCTCCAAGTAC |
| | | ATGATTGTGGTGGGCCAGGAGATTATCTACGACATCCGCAAGGACTTG |
| | | TTCGAGCACCTGCAGAAGCTGCCCTTCCAGTTTTACGATGACCGGCCC |
| | | CACGGCAAGATTTTGACCCGCGTTATCAACTATGTCAACTCCGTGTCG |
| | | GATGCCCTCTCCAACGGCATCATCAACTTTGTGCTGGAGATCTTCAAC |
| | | CTGATCTTGATTGCCGTGTTCATGTTCCTGTGCGATGTGCGCCTGAGC |
| | | CTGATCGTCATGGCGGGCATCCCCCTGTTTCTGGTCATCGTGCTGCTC |
| | | ATCAAGCCCGCCCAGCGCCGGGCCTGGCAGGATGTGTCCAACAAGAGC |
| | | TCCAATATCAACGCCTACCTCCACGAAAGCCTGGACGGCATGAAGATC |
| | | ACCCAGGCCTTCACCCGGGAGGAGGAGAACCGCGGCATCTACGAGAAG |
| | | CTGAACAAGAAGTGCTATCAGACCTGGATGAAGGCTCAGTACACCTCC |
| | | AACCTGGTGTGGTACTCTGTGGACAACATCTCCACCTGGGTGGTGGGC |
| | | GCCATGTACCTCATCGGCCTGTGGATGCTGGGGCCCGCCATGCAGATT |
| | | GGCACCCTCATTGCCATTTCCTCCTACGCTTGGCGGTTCTGGCAGCCC |
| | | ATTTTGAACCTGTCCAACCTGTACAACACCTTCATCAACGCGGTGGCC |
| | | TATCTGGAGCGCATCTTCGAGATGATTGACGAGCCCGTTACTGTGGAC |
| | | GATGCCCCCGGCGCCACCGAGCTGCCCCCCATCACCGGCCGTGTCACC |
| | | TTCGATGACGTGACCTTCTCCTATGATGGGCAAATCAACATCTTAGAG |
| | | CACTTCAACCTGGATGTAAAGCCCGGCGAGTCCATTGCCCTGGTGGGC |
| | | CCCACCGGCGCCGGCAAGACCACTGTAGTGAACTTGATCTCCCGGTTC |
| | | TATAACATCGACAGGGGACGCCTGCTGCTGGATGGCCACGACATCGCC |
| | | CAGGTGACCCTCCGCTCCCTTCGCTCTCAAATGGGCATTATGCTTCAG |
| | | GACAGCTTCATCTTCTCCGGCACCATTATGGACAACATCCGCTATGGC |
| | | CGCCTGGACGCCACCGATGAGGAGGTCATCGCCGCGGCCAAGACCGTG |
| | | CGCGCGGATGAGTTCATCCGGGAAATGGAGGATGGCTATTACACCCAG |
| | | GTCAACGAGCGGGGCTCCCGCCTGTCCCAGGGCCAGCGGCAGCTGGTG |
| | | GCCTTTGCCCGCACCCTGCTCTCCGACCCCAAGATTCTGGTGCTGGAT |
| | | GAGGCCACCTCTTCCATCGACGCCAAAACCGAGCGCCTGGTGCAAGAG |
| | | GGCCTCAACGCCCTTCTGAAAGGCCGTACCAGCTTTATCATCGCCCAC |
| | | CGCCTTTCCACCATCAAGAACTGCGACCGCATCCTGTACATTTCCAAC |
| | | AAGGGCATTGCGGAGATGGGCACCCATCAGCAGCTGTTGGAGAAGAAG |
| | | GGATATTATTACCATCTGTACACGGCCCAGCTGGAGAGCTGA |
| 8 | Nucleotide | ATGTTTCAGTTAAAGTGGGTGTGGAAGCAGATGGAGGGCTTCCGAAAG |
| | encoding | CGGTACATCTTTGCCCTGTTCTCCACGGCCCTGCTGGCGATGCTGACC |
| | MsbA1- | CTGGGCAACTCAGTCATCACCGCCAGCATTATGGACACGGTGTTCCAG |
| | MsbA2-like | CCGCTTACCGAAAGCGGTGTGGTCACCCAGCAGGTGGTGCACCATCTG |
| | | GCGGTTCTGGTTGCGGTGCTCATTGGGTTTACGTTGTTCCGCACCAGC |
| | | TTTCAGTACCTATCCATTATGACCTATGAGGGCTGTTCCCAAAAGCTA |
| | | ATCTTCAAGCTCCGCCGGGATTTGTACAAGAATATGCAGGAGCAGGAC |
| | | CAGGACTTCTTCTCCAAAACCCGCACCGGCGACCTGATGACCCGCCTC |
| | | ACCGGCGACCTGGATATGGTCCGGTTTGCCGTGGCCTGGGTGGTGCGG |
| | | CAGCTGATCCACTGCACAGTGCTGTTTGTCACCACCTCCATTGTGTTT |
| | | TTGGTGACAGACTGGCTCTTTGCCCTGTCCATGCTGGCGGTAACGCCC |
| | | ATTATCTTTGCCCTGACGCTGGCCTTCTCCAAGCGGGTGCACCCCCTG |
| | | TATGTGGACCTGCGGGAGCGCCTCTCTTTGCTAAACACCCAGGCCCAG |
| | | GAGAACATCTCTGGCAACCGGGTGGTAAAGGCCTTCGCCCGGGAGGAC |
| | | TATGAGATCGACCGGTTCGATGAGAAAAATGCCGACTATAAAAAGGCC |
| | | AACACCAGGGCCTCCCTGCTGTGGCTGCAGTACAGCCCCTATATTGAG |
| | | GGCCTGTCCCAGTCCCTCTCCATTGCGGTGCTGCTGGTGGGGGGCGTG |
| | | TTCCTCATCACCGGGCGCATCTCAATTGGGACCTTCACCCTGTTCAAC |
| | | GGCTTGACCTGGACGCTGACCGACCCCATGCGCATGTTGGGCATGCAC |
| | | CTCAACGACCTGCAGCGTTTCTTCGCCAGTTCCAATAAGATTATCGAG |
| | | CTGTACTATGCCAAGTCCACCATCACTTCCCGGCCCGACGCCAAAAAG |
| | | GTGGACACCCGCCTGAAGGGGGAGATCGACTTCTCCGGCGTGGACCTG |
| | | AACCTCCACGGCCAACCGGTGCTGCGCCACATTGACCTGCATATCAAT |
| | | CCTGGCGAAACGGTGGCCATTATGGGGCCCACCGGCTCTGGAAAGACC |
| | | TCGCTGGTGAATTTGATCCCCCGGTTTACGGACGTCAGCGGCGGCACC |
| | | CTCACCATAGACGGCACGCCGGTGGGGCGCTATGACCTGCAGGGCCTG |
| | | CGCCACGCTATCGGTATCGCCACCCAGGACGTGTTCCTGTTTTCCGAC |
| | | ACGGTGGACGGCAATATCGCCTATGGCGACTCCTCCCTCTCCGAGGAT |
| | | GACGTGAAGCGCTATGCCAAAATGGCGGATGTGGACTTTGTGGAAAAG |
| | | CTTCCCGATGGCTTCGACACCCTCATTGGCGAGCGGGGCACCGGCCTT |
| | | TCCGGCGGCCAGAAGCAGCGCATCGCCCTGGCCCGGGCCCTGGCCGTG |
| | | CGGCCCTCCATCCTCATCTTGGATGACACCACCAGCGCCGTGGACCTG |
| | | GAAACCGAGAAGTACATCCAAGAGCAGCTGGCCAGCTTGGACTTCCCC |
| | | TGCACCAAGATCATCGTGGCCCAGCGCATTTCCACCACCAAGCGGGCG |
| | | GATAAGATCGTCATTTTGGATAAGGGCCGGGTAGTGGACATCGGCACC |
| | | CACGAGGAGCTGTCCCAGCGGCCCGGCTACTACCGGGAAGTGTTCCTG |
| | | CTGCAAAACGGCATGGAAGAGGAAAAGGAGGTGGTTTAAATGGCACGC |
| | | AACAAGTTTGACGTGGACGAAACCCTGGAAACCCCATTTAACATAAAG |
| | | CATCTGCTCCGGGCCGGCGTGTACATTGGCCGCCACAAGAAAAAGATG |
| | | ATTTTGTCCCTGCTGTTCTCCGCCATTTCCGCCGCCTGCTCTCTGCTG |
| | | GGGCCCATGCTCATCCAGCGGGCCATTGACGTGTCCGTGCCCCAAAAG |
| | | GACTACACCGAGCTGGTGGTGCTGGCCGTCATCATGCTGGTGTCCATT |
| | | GTGGCCTCTGTGCTGTTCGCCCGGGCCCGCTCCAAGTACATGATTGTG |
| | | GTGGGCCAGGAGATTATCTACGACATCCGCAAGGACTTGTTCGAGCAC |
| | | CTGCAGAAGCTGCCCTTCCAGTTTTACGATGACCGGCCCCACGGCAAG |
| | | ATTTTGACCCGCGTTATCAACTATGTCAACTCCGTGTCGGATGCCCTC |
| | | TCCAACGGCATCATCAACTTTGTGCTGGAGATCTTCAACCTGATCTTG |
| | | ATTGCCGTGTTCATGTTCCTGTGCGATGTGCGCCTGAGCCTGATCGTC |
| | | ATGGCGGGCATCCCCCTGTTTCTGGTCATCGTGCTGCTCATCAAGCCC |
| | | GCCCAGCGCCGGGCCTGGCAGGATGTGTCCAACAAGAGCTCCAATATC |
| | | AACGCCTACCTCCACGAAAGCCTGGACGGCATGAAGATCACCCAGGCC |
| | | TTCACCCGGGAGGAGGAGAACCGCGGCATCTACGAGAAGCTGAACAAG |
| | | AAGTGCTATCAGACCTGGATGAAGGCTCAGTACACCTCCAACCTGGTG |
| | | TGGTACTCTGTGGACAACATCTCCACCTGGGTGGTGGGCGCCATGTAC |
| | | CTCATCGGCCTGTGGATGCTGGGGCCCGCCATGCAGATTGGCACCCTC |
| | | ATTGCCATTTCCTCCTACGCTTGGCGGTTCTGGCAGCCCATTTTGAAC |
| | | CTGTCCAACCTGTACAACACCTTCATCAACGCGGTGGCCTATCTGGAG |
| | | CGCATCTTCGAGATGATTGACGAGCCCGTTACTGTGGACGATGCCCCC |
| | | GGCGCCACCGAGCTGCCCCCCATCACCGGCCGTGTCACCTTCGATGAC |
| | | GTGACCTTCTCCTATGATGGGCAAATCAACATCTTAGAGCACTTCAAC |
| | | CTGGATGTAAAGCCCGGCGAGTCCATTGCCCTGGTGGGCCCCACCGGC |
| | | GCCGGCAAGACCACTGTAGTGAACTTGATCTCCCGGTTCTATAACATC |
| | | GACAGGGGACGCCTGCTGCTGGATGGCCACGACATCGCCCAGGTGACC |
| | | CTCCGCTCCCTTCGCTCTCAAATGGGCATTATGCTTCAGGACAGCTTC |
| | | ATCTTCTCCGGCACCATTATGGACAACATCCGCTATGGCCGCCTGGAC |
| | | GCCACCGATGAGGAGGTCATCGCCGCGGCCAAGACCGTGCGCGCGGAT |
| | | GAGTTCATCCGGGAAATGGAGGATGGCTATTACACCCAGGTCAACGAG |
| | | CGGGGCTCCCGCCTGTCCCAGGGCCAGCGGCAGCTGGTGGCCTTTGCC |
| | | CGCACCCTGCTCTCCGACCCCAAGATTCTGGTGCTGGATGAGGCCACC |
| | | TCTTCCATCGACGCCAAAACCGAGCGCCTGGTGCAAGAGGGCCTCAAC |
| | | GCCCTTCTGAAAGGCCGTACCAGCTTTATCATCGCCCACCGCCTTTCC |
| | | ACCATCAAGAACTGCGACCGCATCCTGTACATTTCCAACAAGGGCATT |
| | | GCGGAGATGGGCACCCATCAGCAGCTGTTGGAGAAGAAGGGATATTAT |
| | | TACCATCTGTACACGGCCCAGCTGGAGAGCTGA |
| 9 | Polypeptide | MFQLKWVWKQMEGFRKRYIFALFSTALLAMLTLGNSVITASIMDTVFQ |
| | MsbA1- | PLTESGVVTQQVVHHLAVLVAVLIGFTLFRTSFQYLSIMTYEGCSQKL |
| | MsbA2-like | IFKLRRDLYKNMQEQDQDFFSKTRTGDLMTRLTGDLDMVRFAVAWVVR |
| | | QLIHCTVLFVTTSIVFLVTDWLFALSMLAVTPIIFALTLAFSKRVHPL |
| | | YVDLRERLSLLNTQAQENISGNRVVKAFAREDYEIDRFDEKNADYKKA |
| | | NTRASLLWLQYSPYIEGLSQSLSIAVLLVGGVFLITGRISIGTFTLFN |
| | | GLTWTLTDPMRMLGMHLNDLQRFFASSNKIIELYYAKSTITSRPDAKK |
| | | VDTRLKGEIDFSGVDLNLHGQPVLRHIDLHINPGETVAIMGPTGSGKT |
| | | SLVNLIPRFTDVSGGTLTIDGTPVGRYDLQGLRHAIGIATQDVFLFSD |
| | | TVDGNIAYGDSSLSEDDVKRYAKMADVDFVEKLPDGFDTLIGERGTGL |
| | | SGGQKQRIALARALAVRPSILILDDTTSAVDLETEKYIQEQLASLDFP |
| | | CTKIIVAQRISTTKRADKIVILDKGRVVDIGTHEELSQRPGYYREVFL |
| | | LONGMEEEKEVVMARNKFDVDETLETPFNIKHLLRAGVYIGRHKKKMI |
| | | LSLLFSAISAACSLLGPMLIQRAIDVSVPQKDYTELVVLAVIMLVSIV |
| | | ASVLFARARSKYMIVVGQEIIYDIRKDLFEHLQKLPFQFYDDRPHGKI |
| | | LTRVINYVNSVSDALSNGIINFVLEIFNLILIAVFMFLCDVRLSLIVM |
| | | AGIPLFLVIVLLIKPAQRRAWQDVSNKSSNINAYLHESLDGMKITQAF |
| | | TREEENRGIYEKLNKKCYQTWMKAQYTSNLVWYSVDNISTWVVGAMYL |
| | | IGLWMLGPAMQIGTLIAISSYAWRFWQPNLSNLYNTFINAVAYLERIF |
| | | EMIDEPVTVDDAPGATELPPITGRVTFDDVTFSYDGQINILEHFNLDV |
| | | KPGESIALVGPTGAGKTTVVNLISRFYNIDRGRLLLDGHDIAQVTLRS |
| | | LRSQMGIMLQDSFIFSGTIMDNIRYGRLDATDEEVIAAAKTVRADEFI |
| | | REMEDGYYTQVNERGSRLSQGQRQLVAFARTLLSDPKILVLDEATSSI |
| | | DAKTERLVQEGLNALLKGRTSFIIAHRLSTIKNCDRILYISNKGIAEM |
| | | GTHQQLLEKKGYYYHLYTAQLES |

Vectors and Cassettes of the Invention

In another aspect, the present invention also relates to expression of non-natural vectors and cassettes that allow the expression of the recombinant heterodimeric protein of the invention. These vectors and cassettes comprise the recombinant nucleic acid(s) as defined above. Preferably, they are capable of transforming host cells such as bacterial cells, where the heterodimeric (MsbA1-MsbA2-like transporter) protein of the invention is preferably expressed, in order to promote the secretion of the anti-inflammatory muropeptide precursors.

In a preferred embodiment, these vectors and expression cassettes express or encode a heterodimeric protein whose first and second polypeptides have a sequence of at least 80%, at least 85%, at least 90%, or at least 95% sequence identity with SEQ ID NO:1 and SEQ ID NO:2 respectively and have the same biological function as SEQ ID NO:1 and SEQ ID NO:2 respectively. In another preferred embodiment, these vectors and expression cassettes express or encode a heterodimeric protein having a sequence of at least 80%, at least 85%, at least 90%, or at least 95% sequence identity with SEQ ID NO:9 and having the same biological function as SEQ ID NO:9 does.

In a more preferred embodiment, these vectors and expression cassettes contain the recombinant nucleic acid having the sequence SEQ ID NO:3 and/or the recombinant nucleic acid having the sequence SEQ ID NO:4, or homologous sequences thereof. These two polynucleotides are preferably located in the same Open Reading Frame. More preferably, they are consecutively located, as depicted in SEQ ID NO:8. These homologous sequences of SEQ ID NO:3 and 4 preferably express or encode the same functional proteins SEQ ID NO:1 or SEQ ID NO:2 or homologous thereof having the same biological function.

In an even more preferred embodiment, the vectors and expression cassettes contain the recombinant nucleic acid comprising or having the sequence SEQ ID NO:5, as depicted in the enclosed listing, or an homologous sequence thereof. SEQ ID NO:5 contains SEQ ID NO:3, SEQ ID NO:4 and naturally occurring regulatory sequences and ORFs that may have an influence on the expression of the heterodimeric protein and on the availability of the muropeptide precursors transported by said protein.

Any non-natural or natural vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (e.g., expression of heterologous polynucleotide) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence.

Said vectors or cassettes of the invention preferably contain regulatory sequences allowing the expression of the polypeptides of the invention in an host cell, preferably in a bacterial host cell, e.g., a Escherichia coli Gram-negative bacterium. These regulatory sequences are for example a promoter, which is inducible or constitutive. Promoters suitable for use with prokaryotic hosts include E. coli promoters such as lac, trp, tac, trc and ara, viral promoters recognized by E. coli such as lambda and T5 promoters, and the T7 and TTlac promoters derived from T7 bacteriophage.

In a preferred embodiment, the recombinant nucleotide of the invention contains promoter that is not naturally associated with SEQ ID NO:3 and SEQ ID NO:4. More precisely, the recombinant nucleotide of the invention preferably does not contain the natural promoter associated with SEQ ID NO:3 and SEQ ID NO:4 in a Firmicutes Gram-positive bacterium. It can however contain any other promoter that is efficient in a host cell, and more precisely in bacteria.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using a pBR322, pUC, pET or pGEX vector, a plasmid derived from an E. coli species. Such vectors contain genes encoding ampicillin (Amp) and tetracycline (Tet) resistance and thus provides easy means for identifying transformed cells. These vectors as well as their derivatives or other microbial plasmids or bacteriophage may also contain, or be modified to contain, promoters which can be used by the microbial organism for expression of endogenous proteins.

Suitable heterologous vectors for expression in both prokaryotic and eukaryotic host cells are known in the art and some are further described herein.

An expression vector of the present disclosure may comprise a promoter as described above, as well as a signal sequence which allows the translated recombinant protein to be recognized and processed (i.e., cleaved by a signal peptidase) by the host cell.

As meant in the present invention, an expression cassette is a component of vector DNA consisting of a gene and regulatory sequence to be expressed by a transfected cell. In each successful transformation, the expression cassette directs the cell's machinery to make RNA and protein(s). Some expression cassettes are designed for modular cloning of protein-encoding sequences so that the same cassette can easily be altered to make different proteins. An expression cassette is typically composed of one or more genes and the sequences controlling their expression. An expression cassette comprises usually at least three components: a promoter sequence, an open reading frame, and a 3′ untranslated region that, in eukaryotes, usually contains a polyadenylation site. Different expression cassettes can be transfected into different organisms including bacteria, yeast, plants, and mammalian cells as long as the correct regulatory sequences are used. These regulatory sequences are well-known in the art.

Recombinant vectors or cassettes may remain free (i.e., non-integrated into a host cell genome), or may become integrated (“stably” or “temporary” incorporated) into the host cell genome, either as a result of the original transformation of the host cells, or as the result of subsequent recombination and/or repair events. Still, they remain “heterologous” as compared to the host cell and its natural genome.

Host Cells of the Invention

As mentioned previously, the present inventors have shown that bacteria cells that have been genetically modified so as to express the heterodimeric transporter of the invention are able to secrete bioactive anti-inflammatory compounds (notably muropeptide precursors) that protect the epithelial barrier from bacterial infection and associated inflammation. These genetically engineered bacteria are therefore a promising tool for preventing or treating disorders are associated with decreased gastrointestinal epithelial cell barrier function or integrity, such as IBDs.

Therefore, in another aspect, the present invention relates to a recombinant host cell, typically a bacterium, comprising a recombinant nucleic acid expressing an MsbA1-MsbA2-like transporter, more particularly the expression vector of the invention, or the expression cassette of the invention, comprising said recombinant nucleic acid expressing an MsbA1-MsbA2-like transporter, as defined above.

As used herein, the term “recombinant host cell” more generally refers to any cell or cell line into which a recombinant expression vector for production of a polypeptide can be introduced and expressed.

In a preferred embodiment, these host cells therefore express at their membrane the heterodimeric protein whose first and second polypeptides have the sequences SEQ ID NO:1 and SEQ ID NO:2, respectively.

In a more preferred embodiment, these recombinant host cells contain the recombinant nucleic acid having the sequence SEQ ID NO:3 and/or the recombinant nucleic acid having the sequence SEQ ID NO:4, or homologous sequences thereof, these two polynucleotides being preferably located in the same ORF (SEQ ID NO:8).

In a more preferred embodiment, these recombinant host cells contain the recombinant vectors or cassettes of the invention, as defined above. They can contain either a unique vector/cassette encoding the two polypeptides of the invention, or two distinct vectors/cassettes, each of them encoding one of the two polypeptides of the invention. It is herein preferred that the host cells of the invention contain a unique recombinant vector/cassette expressing the two polypeptides of the invention.

In an even more preferred embodiment, these recombinant host cells contain the recombinant polynucleotide comprising or having the sequence SEQ ID NO:5 (or an homologous sequence thereof), as depicted in the enclosed listing. In a very particular embodiment, the recombinant host cells contain only the polynucleotide of SEQ ID NO:5 (or an homologous sequence thereof), as a source of the heterodimeric protein of the invention.

Preferably, the recombinant host cell of the invention is a genetically engineered prokaryotic bacterium.

More preferably, this recombinant bacterium is non-pathogenic, so as to be safely administered in humans.

“Non-pathogenic bacteria” refer to bacteria that are not capable of causing disease or harmful responses in a host. In some embodiments, non-pathogenic bacteria are commensal bacteria. Examples of non-pathogenic bacteria include, but are not limited to: Bacillus, Bacteroides, Bifidobacterium, Brevibacteria, Clostridium, Enterococcus, Escherichia coli, Lactobacillus, Lactococcus and Saccharomyces, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii.

In a preferred embodiment, this host cell is a non-pathogenic Gram-negative bacterium, for example a proteobacteria including Escherichia coli (E. coli). As a matter of fact, the muropeptide precursors transported and described herein/below by the transporter of the invention are produced in Gram-negative bacteria. The examples below disclose that the E. Coli Epi300 can secrete higher amounts of these muropeptide precursors, when transfected by the vector of the invention.

In a preferred embodiment, the present invention relates to recombinant Escherichia Coli host cells containing the recombinant vector of the invention, as described above. In particular, these recombinant Escherichia Coli host cells contain an heterologous nucleic acid encoding a heterodimeric protein comprising a first polypeptide whose amino acid sequence is SEQ ID NO: 1 or a homologous sequence thereof having the same biological function, and a second polypeptide whose amino acid sequence is SEQ ID NO: 2 or a homologous sequence thereof having the same biological function. These recombinant Escherichia Coli host cells therefore artificially express the heterodimeric protein of the invention (SEQ ID NO:1 and SEQ ID NO:2), facilitating the secretion of anti-inflammatory muropeptide precursors in the extracellular medium.

Methods for introducing vectors and producing proteins are well known to the ordinarily skilled artisan. The host cells are first transformed or transfected with expression vectors or cassettes as those described above, by conventional means (e.g., electroporation). Then protein production is induced by culturing the cells in conventional nutrient media that has been chosen or modified so as to inducing promoters, selecting and/or maintaining transformants, and/or expressing the genes encoding the desired protein sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 19 1) and Molecular Cloning: A Laboratory Manual (Sambrook, et al, 1989, Cold Spring Harbor Laboratory Press).

Particular transfer methods, such as phages, plasmids, and transposons, can be used to deliver and circulate engineered DNA sequences to microbial communities, via processes such as transduction, transformation, and conjugation. An engineered phage could be one possible delivery system for a protein of the disclosure, by incorporating the nucleic acid encoding said protein into the phage and utilizing the phage to deliver the nucleic acid to a host microbe that would then produce the protein after having the phage deliver the nucleic acid into its genome.

One could also utilize a transposon delivery system to incorporate nucleic acids encoding a therapeutic protein into a host microbe that is resident in a patient's microbiome (Sheth, et al., Trends in Genetics, 2016, Vol. 32, Issue 4, pgs, 189-200). In this case, the host cell of the invention is therefore a bacterium that is part of the microbiome.

One particular recombinant bacterial delivery system is based upon Escherichia coli bacteria. Essentially, one may clone the gene encoding the therapeutic protein (e.g. SEQ ID NO:3 and 4 or or 8) into an expression vector, and then transform said vector into E. coli. Subsequently, one may then administer the E. coli to a patient.

In another embodiment, a “synthetic bacterium” may be used as a probiotic bacterium engineered to express the heterodimeric protein of the invention (see, e.g., Durrer and Allen, 2017, PLoS One, 12:e0176286).

Muropeptide Precursors of the Invention

The purification of the two active fractions resulted in the identification of the following muropeptide precursors:

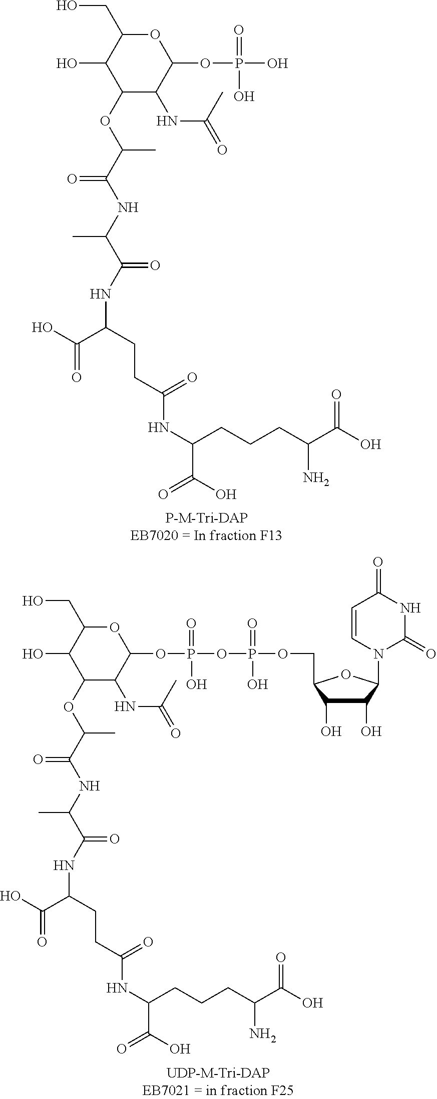

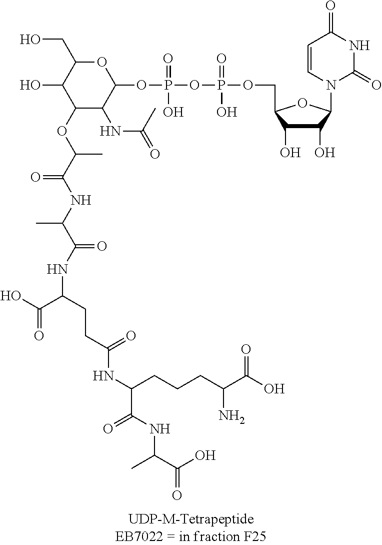

EB7020 is a P-M-TriDAP ou M-Tri-DAP monophosphate having the formula C₂₆H₄₄N₅O₁₈P. Its exact mass is 745,2419; its Molecular weight is of 745,6240.

EB7021 is a UDP-M-Tri-DAP having the formula C₃₅H₅₅N₇O₂₆P₂. Its exact mass is 1051,267; its Molecular weight is of 1051,790.

EB7022 is a UDP-M-Tetrapeptide (UDP-M-TetraDAP) having the formula C₃₈H₆₀N₈O₂₇P₂. Its exact mass is 1122,304; its Molecular weight is of 1122,868.

As shown in the examples below, these muropeptide precursors participate in the protective effect of the epithelium which is specifically observed in inflammatory circumstances.

The present invention therefore relates to any of these muropeptide precursors, or at least two of said three muropeptide precursors, or the three of them, or to a pharmaceutical composition containing same, for use as a medicament, for example for:
reducing gastrointestinal inflammation in a patient in need thereof,
reducing intestinal mucosal inflammation in a patient in need thereof,
increasing gastrointestinal wound healing in a patient in need thereof,
increasing intestinal epithelial cell proliferation in a patient in need thereof, or for
treating or preventing epithelial barrier function disorders, in a patient in need thereof.

Preferably, the pharmaceutical composition of the invention contains a mixture of the three muropeptide precursors, typically between 1 nM and 10 μM, preferably between 5 nM and 5 μM, more preferably between 10 nM and 1 μM, even more preferably between 50 nM and 0.5 μM of each muropeptide precursor.

Pharmaceutical Compositions of the Invention

In other aspects, the present invention provides a pharmaceutical composition comprising a therapeutically effective amount of the host cell of the invention, or of the muropeptide precursor(s) of the invention, as described above, and a pharmaceutically acceptable carrier.

This pharmaceutical composition can be formulated for rectal, parenteral, intravenous, topical, oral, dermal, transdermal, or subcutaneous administration.

In a particular embodiment, the pharmaceutical composition of the invention is a capsule, a liquid, a gel, an emulsion or a cream. In another embodiment, the pharmaceutical composition is a solid composition comprising an enteric coating, so as to delay the release into the gastrointestinal tract, e.g., until the small intestine or the large intestine or the rectum, preferably over a time period of about 1 to 20 hours, 1 to 10 hours, 1 to 8 hours, 4 to 12 hours or 5 to 15 hours.

As used herein, the term “therapeutically effective amount” refers to an amount of a therapeutic agent (e.g., the host cell of the disclosure or the muropeptide precursors of the invention), which confers a therapeutic effect on the treated subject, at a reasonable benefit/risk ratio applicable to any medical treatment. Such a therapeutic effect may be objective (i.e., measurable by some test or marker) or subjective (i.e., the subject gives an indication of, or feels an effect). In some embodiments, a “therapeutically effective amount” refers to an amount of a therapeutic agent or composition that is effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the particular form of disease being treated; the severity of the condition or pre-condition; the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

The present invention uses therapeutically effective amounts of recombinant bacteria, or of muropeptide(s) and compositions comprising same, to treat a variety of diseases, such as gastrointestinal inflammatory diseases or diseases involving gastrointestinal epithelial barrier malfunction. The therapeutically effective amounts of the cells, or muropeptide precursors, or compositions comprising same, will in some embodiments reduce inflammation associated with IBD or repair gastrointestinal epithelial barrier integrity and/or function.

The term “pharmaceutical” herein implies that a composition, reagent, method, and the like, are capable of a pharmaceutical effect, and also that the composition is capable of being administered to a subject safely. “Pharmaceutical effect,” without limitation, can imply that the composition, reagent, or method, is capable of stimulating a desired biochemical, genetic, cellular, physiological, or clinical effect, in at least one individual, such as a mammalian subject, for example, a human, in at least 5% of a population of subjects, in at least 10%, in at least 20%, in at least 30%, in at least 50% of subjects, and the like.

The term “pharmaceutically acceptable” herein means that said feature has been approved by a regulatory agency of the Federal or a state government or listed in recognized pharmacopoeia for safe use in animals, and more particularly safe use in humans. A “pharmaceutically acceptable carrier” refers to a diluent, adjuvant, excipient or carrier with which the host cells of the invention, as described above, can be safely and efficiently administered.

Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohol such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the cells of the invention, or of the pharmaceutical compositions containing same.

In some embodiments, the pharmaceutical composition of the invention further comprises a second therapeutic agent. This second therapeutic agent is for example selected from the group consisting of an anti-diarrheal, a 5-aminosalicylic acid compound, an anti-inflammatory agent, an antibiotic, an anti-cytokine agent, an anti-inflammatory cytokine agent, a steroid, a corticosteroid, an immunosuppressant, a JAK inhibitor, an anti-integrin antibody, an anti-IL12/23R antibody, and a vitamin.

In a particular embodiment, said second therapeutic agent can be an aminosalicylate, a steroid, a corticosteroid, or an agent selected from the group consisting of adalimumab, pegol, golimumab, infliximab, vedolizumab, ustekinumab, tofacitinib, and certolizumab or certolizumab pegol.

The therapeutic pharmaceutical compositions taught herein may comprise one or more natural products. However, the therapeutic pharmaceutical compositions, containing recombinant cells that have been engineered so as to overexpress particular polypeptides, do not occur in nature. In other words, the host cells of the invention, as well as the therapeutic pharmaceutical compositions of the invention, possess markedly different characteristics, as compared to naturally occurring bacteria or compositions.

Methods of Treatment/Therapeutic Uses of the Invention

As explained thoroughly above, the present invention relates to the use of the host cell as described above, or of the muropeptide precursor(s) of the invention, or of the pharmaceutical composition containing same, as described above, as a medicament.

Recent studies have identified a major role of both genetic and environmental factors on the pathogenesis of IBD (MF Neurath, Nature Reviews Immunology, 2014 Vol. 14., 329-342). A combination of these IBD risk factors seems to initiate detrimental changes in epithelial barrier function, thereby allowing the translocation of luminal antigens (for example, bacterial antigens from the commensal microbiota) into the bowel wall. Subsequently, aberrant and excessive responses, such as increased pro-inflammatory cytokine release cause subclinical or acute mucosal inflammation in a genetically susceptible host. Moreover, the importance of a functional epithelial barrier in IBD is apparent, for in subjects that fail to resolve acute intestinal inflammation, chronic intestinal inflammation develops, induced by the uncontrolled activation of the mucosal immune system. In particular, mucosal immune cells, such as macrophages, T cells, and the subsets of innate lymphoid cells (ILCs), can respond to microbial products or antigens from the commensal microbiota by producing cytokines that can promote chronic inflammation of the gastrointestinal tract.

Moreover, there are numerous other diseases that have been shown to be caused, linked, correlated, and/or exacerbated by, an improperly functioning epithelial barrier. These diseases include: (1) metabolic diseases, including obesity, type 2 diabetes, non-alcoholic steatohepatitis (NASH), non-alcoholic fatty liver disease (NAFLD), liver disorders, and alcoholic steatohepatitis (ASH); (2) celiac disease; (3) necrotizing enterocolitis; (4) irritable bowel syndrome (IBS); (5) enteric infections (e.g. Clostridium difficile); (6) other gastro intestinal disorders in general; (7) interstitial cystitis; (8) neurological disorders or cognitive disorders (e.g. Alzheimer's, Parkinson's, multiple sclerosis, and autism); (9) chemotherapy associated steatohepatitis (CASH); and (10) paediatric versions of the aforementioned diseases (Everard et al, Diabetes, Vol. 60, 2011, pgs. 2775-2786; Everard et al, PNAS, Vol. 1 10, No. 22, 2013, pgs. 9066-9071; Cam et al, Diabetes, Vol. 57, 2008, pgs. 1470-1481; Delzenne et al., Nature Reviews, Vol. 7, 2011, pgs. 639-646. Consequently, restoring proper epithelial barrier function to patients may be critical in resolving the aforementioned disease states.

The therapeutic activity of the sequences of the invention was identified in part by their beneficial effects on epithelial barrier function ex vivo on ileal explants confronted to live pathogenic bacteria. As shown in FIG. 5, the supernatant of the recombinant bacteria of the present invention (comprising SEQ ID NO:3 and 4) increased epithelial barrier integrity as shown in an in vitro trans-epithelial electrical resistance (TEER) assay. TEER assays are well-known methods for measuring effects on the structural and functional integrity of an epithelial cell layer (Srinivasan et ai., 2015, j Lab Autom, 20: 107-126).

Furthermore, array study on DCs showed that HPLC-purified fractions F13 and F25 upregulate genes linked to epithelial barrier (Table 2 below) while downregulating pro-inflammatory genes (Table 3 below). Indeed, expression of tight junctions genes, involved in maintaining epithelial integrity, was upregulated on HT-29 epithelial cells stimulated with pre-purified supernatant of F4 clone (FIG. 4).

Furthermore, as shown on FIGS. 1, 3, 8 and 9, the clone and the muropeptide precursors of the invention are able to promote the secretion of the anti-inflammatory cytokine IL-10.

This means that the bioactive compounds present in the supernatant of this clone have a protective role on the epithelial barrier. This has been confirmed ex vivo on FIG. 6.

In a preferred embodiment, the host cell and the pharmaceutical composition of the invention are thus useful for increasing the barrier function of the epithelial cell layer, for increasing the secretion of anti-inflammatory cytokines such as IL-10.

They can therefore be used in vivo for increasing intestinal epithelial cell wound healing or for reducing the intestinal tissue pathology, specifically the gastrointestinal mucosa inflammation in a subject in need thereof, e.g., in a subject having intestinal tissue damages due to a treatment with a chemical.

It is therefore contemplated to use the medicament of the invention for either:
reducing gastrointestinal inflammation in a patient in need thereof, or for
reducing intestinal mucosal inflammation in a patient in need thereof, or for
increasing gastrointestinal wound healing in a patient in need thereof, or for
increasing intestinal epithelial cell proliferation in a patient in need thereof, or for
treating or preventing epithelial barrier function disorders, in a patient in need thereof.

Said epithelial barrier function disorder can be for example chosen in the group consisting of: inflammatory bowel disease, ulcerative colitis, pediatric UC, Crohn's disease, pediatric Crohn's disease, short bowel syndrome, mucositis GI mucositis, oral mucositis, mucositis of the esophagus, stomach, small intestine (duodenum, jejunum, ileum), large intestine (colon), and/or rectum, chemotherapy-induced mucositis, radiation-induced mucositis, necrotizing enterocolitis, pouchitis, a metabolic disease, celiac disease, irritable bowel syndrome, or chemotherapy associated steatohepatitis (CASH).

In a preferred embodiment, said epithelial barrier function disorder is an IBD, such as the Crohn Disease. In another preferred embodiment, said disorder is ulcerative colitis.

As used herein, the term “treatment” (also “treat” or “treating”) refers to any administration of a therapeutic agent (e.g., the host cell of the disclosure), according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., chronic or recurring immune response and inflammation of the gastrointestinal (GI) tract); in some embodiments, administration of the therapeutic agent according to the therapeutic regimen is correlated with the achievement of the desired effect.

“Preventing” or “prevention” refers to the reduction of the risk of acquiring a disease or disorder in a subject that may be exposed to or predisposed to the disease but does not yet experience or display symptoms of the disease. “Prevention” or “prophylaxis” may refer to delaying the onset of the disease or disorder.

The terms “patient,” “subject,” and “individual” may be used interchangeably and refer to either a human or a non-human animal. These terms include mammals such as humans, non-human primates, livestock animals, companion animals (e.g., canines, felines) and rodents (e.g., mice and rats). In certain embodiments, the terms refer to a human patient. In a preferred embodiment, the terms refer to a human patient that suffers from a gastrointestinal inflammatory condition.

In a particular embodiment, the subject in need thereof has been diagnosed with intestinal inflammation, e.g., in the small intestine and/or the large intestine and/or in the rectum.

In another particular embodiment, the subject in need thereof has been diagnosed with intestinal ulcers.

In a particular embodiment, the subject in need thereof has been diagnosed with Crohn's disease (CD). In an embodiment, the CD is mildly active CD. In another embodiment, the CD is moderately to severely active CD. In another embodiment, the subject in need thereof has been diagnosed with paediatric CD.

In a particular embodiment, the subject in need thereof has been diagnosed with short bowel syndrome or with irritable bowel syndrome or with mucositis. In particular embodiments, the mucositis is oral mucositis. In still other embodiments, the mucositis is chemotherapy-induced mucositis, radiation therapy-induced mucositis, chemotherapy-induced oral mucositis, or radiation therapy-induced oral mucositis. In yet other embodiments, the mucositis is gastrointestinal mucositis. In still other embodiments, the gastrointestinal mucositis is mucositis of the small intestine, the large intestine, or the rectum.

In a particular embodiment, the subject in need thereof has been diagnosed with ulcerative colitis (UC). In an embodiment, the UC is mildly active UC. In another embodiment, the UC is moderately to severely active UC. In another embodiment, the subject in need thereof has been diagnosed with paediatric UC.

In particular embodiments, the subject in need thereof is in clinical remission from an IBD. In other embodiments, the subject in need thereof is in clinical remission from UC, paediatric UC, CD or paediatric CD.

The present invention also relates to methods for preventing or treating an epithelial barrier function disorder in a subject in need thereof, said methods comprising administering to said subject the host cells of the invention of the pharmaceutical compositions containing same, as described above.

In some embodiments, the administering reduces gastrointestinal inflammation and/or reduces intestinal mucosa inflammation associated with inflammatory bowel disease in the patient. In other embodiments, the administering improves intestinal epithelial cell barrier function or integrity in the patient.

In some embodiments, after the administering, the patient experiences a reduction in at least one symptom associated with said epithelial barrier function disorder. In other embodiments, the at least one symptom is selected from the group consisting of abdominal pain, blood in stool, pus in stool, fever, weight loss, frequent diarrhoea, fatigue, reduced appetite, nausea, cramps, anaemia, tenesmus, and rectal bleeding. In still other embodiments, after the administering, the patient experiences reduced frequency of diarrhoea, reduced blood in stool and/or reduced rectal bleeding.

In some embodiments, the patient has experienced inadequate response to a conventional therapy performed previously to said administering step. This conventional therapy can be a treatment with an aminosalicylate, a corticosteroid, a thiopurine, methotrexate, a JAK inhibitor, a sphingosine 1-phosphate (SIP) receptor inhibitor, an anti-integrin antibody, an anti-IL12/23R or anti-IL23/p40 antibody, and/or an anti-tumor necrosis factor agent or antibody.

Such treatment may be administered to a subject who does not exhibit signs of the relevant disease, disorder and/or condition and/or to a subject who exhibits only early signs of the disease, disorder, and/or condition. Alternatively or additionally, such treatment may be administered to a subject who exhibits one or more established signs of the relevant disease, disorder and/or condition. In some embodiments, treatment may be administered to a subject who has been diagnosed as suffering from the relevant disease, disorder, and/or condition. In some embodiments, treatment may be administered to a subject known to have one or more susceptibility factors that are statistically correlated with increased risk of development of the relevant disease, disorder, and/or condition.

In a final aspect, the present invention relates to the use of the host cell of the invention or of the muropeptide precursors of the invention so as to prepare a medicament which is to be administered to patients suffering from gastrointestinal inflammation, intestinal mucosal inflammation, a gastrointestinal wound or from an epithelial barrier function disorder, as detailed above.

### FIGURE LEGENDS

FIG. 1: AlphaLISA. The supernatant from the complemented F4D5:MsbA1-A2 clone (A1A2) is able to stimulate IL10 secretion from DCs (results obtained using whole supernatants).

FIG. 2: Fractionation of the supernatant of the clone F4 on HPLC (HyperCarb column). Fractions were recovered, dried to eliminate acetonitrile, resuspended in water and tested on HEK Null NF-kB reporter cells.

FIG. 3: AlphaLISA. F4 fractions positive on HEK Null NF-kB reporter cells (cf. FIG. 2) were tested by Alpha LISA on IL10 secretion by DCs.

FIG. 4: Gene expression analysis on HT-29 cells treated with the HPLC-HILIC fraction of F4D5:A1A2 (positive clone) and F4D5-pBAD (negative control). TNFα (10 ng/ml), LPS (10 ng/ml), Na-Butyrate (2 mM).

FIG. 5: TEER measurement 4H post-infection. F−=Fraction from negative control (FADS-pBAD); F+=Fraction from positive F4D5:MsbA1-A2 (A1A2).

FIG. 6: Hematoxylin and eosin (H&E) staining at Objective ×40, on tissues infected with LF82-GFP bacteria, treated or not with F− or F+. F−=Fraction from negative control (pBAD); F+=Fraction from positive F4D5:MsbA1-A2 (A1A2).

FIG. 7: A. Transformed Epi300 bacteria with A1A2 transporter secrete EB7020/EB7021/EB7022 muropeptide precursors very efficiently (10 μM range). B. LC-MS analysis of the supernatant of the clone F4.

FIG. 8: Chemically synthesized EB7020 (F13) generates IL-10 secretion from human DCs.

FIG. 9: Synergy between TLR activators and EB7020/EB7021/EB7022 in generating IL10. LPS 100 ng/ml, FIMH 10 μg/ml, Mean of 3 MoDC donors

### EXAMPLES

1. Material and Methods

1.1 Cell Culture

HEK Null NF-κB/SEAP reporter cells (Invivogen) were used to follow-up the purification of target compounds. The cells were maintained in RPMI 1640 medium (Sigma) with 2 mM L-glutamine, 50 IU/mL penicillin, 50 mg/mL streptomycin, 10 mM Hepes and 10% heat-inactivated foetal calf serum (FCS-Lonza) in a humidified 5% CO2 atmosphere at 37° C.

In activation tests, 30,000 reporter cells/well were seeded in RPMI in 96 wells plates 24h before activation (and kept in a humidified 5% CO2 atmosphere at 37° C.). Pre-purified, or HPLC-eluted, fractions were added to reporter cells at 10% vol/vol to a final volume of 100 μl. SEAP in the supernatant was revealed 24 h after cell stimulation using Quanti-Blue™ reagent (Invivogen) according to the manufacturer's protocol and quantified as OD at 655 nm. Measurements were performed using a Epoch microplate reader (Biotek). Cytotoxicity of target compounds during the purification process was measured (when appropriate) using the The CellTiter 96® AQueous One Solution Cell Proliferation Assay (Promega).

1.2 AlphaLISA

Dendritic Cells (DCs) were prepared from PBMC (Buffy Coat) and differentiated with GM-CSF (D 1:10000; =5 μl/50 ml) and IL-4 (D 1:5000; =10 μl/50 ml).

In this test, 30,000 cells/well were seeded in complete IMDM media (2 mM L-glutamine, 50 IU/mL penicillin, 50 mg/mL streptomycin, 10 mM Hepes and 10% heat-inactivated foetal calf serum) in 96 wells plates 24h before activation (and kept in a humidified 5% CO2 atmosphere at 37° C.). Pre-purified, or HPLC-eluted, fractions as well as EB7020 chemically synthetized and various controls were added to reporter cells at 10% vol/vol to a final volume of 100 μl.

AlphaLisa (Perkin Elmer) was realized on supernatant according the supplier protocol (for complete references: AlphaLISA® Research Reagents Human Interleukin 10 (IL10) Kit Product No.: AL218 C/F sold by PerkinElmer).

1.3. Cloning of MsbA1-A2: Primer List

Target genes were amplified using Phusion High Fidelity DNA Polymerase (New England BioLabs) and primers A1EcoRfw and A2Xbarv (MsbA1-A2 gene).

A1EcoRfw
(SEQ ID NO: 6)
(TTTTGAATTCTTTAGGAGGttttttatgtttcagttaaagtgggtgtgg
aagcag)
A2XbArv
(SEQ ID NO: 7)
(TTTTTCTAGAtcagctctccagctgggccgtgtacagatggtaataata
tc)

Said forward primer contains the AGGAGG RBS consensus sequence.

1.4. Complementation of F4D5 Mutation (Epi300 Transformation)

E. coli Epi300 electrocompetent cells were co-transformed with F4D5 fosmid together with pBAD-MsbA1-A2 plasmid by electroporation. Transformants were selected onto LB agar plates containing 12.5 ug/ml Chloramphenicol, 50 ug/ml Kanamycin and 100 ug/ml Ampicillin.

1.5. Array (DCs)

DCs from three different donors were seeded at 1.0×10⁵ DCs/well in a 48 well plate in 0.5 ml of complete IMDM. The effect of the active fractions of the F4 clone was compared to the same fractions purified from the negative control (pBAD-fractions). TNF-α (10 ng/ml), PHA (3 μg/ml), LPS (3 ng/ml), PAM3CSK4 (10 ng/ml), and MDP 5 μg/ml were used as control.

Data were generated and analyzed using Spotfire software.

1.6. RT-Q-PCR

HT-29 were seeded on 48 wells plate at 150,000 cells/well in 0.5 ml of complete RPMI. Cells were stimulated in a final volume of 0.3 ml with 30 ul of 10× agonists (or pre-purified fractions) for 24 h with: TNF-α (10 ng/ml), LPS (10 ng/ml), Na-butyrate (2 mM), F4 and pBAD-F4 HPLC-Hilic fraction. Before lysis (0.35 ml/well RTL buffer, Qiagen) cells were washed with PBS.

RNA was extracted using the Rneasy minikit (Qiagen). RNA was eluted in 20 μl of MQ water and quantified by Qubit. Reverse Transcription was performed using the High-Capacity cDNA Reverse Transcription Kit (Applied Biosystem) using 1 μg RNA/reaction.

Data were analyzed through the Bio-Rad CFX Manager 3.1 software.

1.7. Ex Vivo Experiments

Human ileal explants were mounted on Ussing chambers and left untreated or pre-treated for 1 h at 37° C. with:
pre-purified fractions of F4D5:MsbA1-A2 clone (designed as F+ in the “Result” section)
pre-purified fractions of its negative control F4D5-pBAD (designed as F− in the “Result” section)

After the 1 h pre-incubation period at 37° C. in a CO2 incubator bacteria were added into the apical compartment of the Ussing chambers at 4×10⁷ and 1×10⁹ bacteria/mL for Salmonella typhimurium and E. coli LF82-gfp, respectively.

The integrity/tightness of the human explants during the incubation was evaluated through Transepithelial Electrical Resistance (TEER) measurement using Millicell-ERS (Electrical Resistance System) Voltohmmeter (Millipore).

At the end of the incubation (6 h for Patient 1 and 4 h for Patient 2), human explants were washed 6-times with 2 mL of PBS, were removed from Ussing chambers and were fixed using PBS PFA 4% solution at 4° C. during 24 h. Fixed human explants were then processed for microscopy and histological analysis (H&E staining).

1.8. LC-MS Analysis

The analysis was carried out using a UPLC system (Vanquish, Thermo Fisher Scientific) coupled with a high-resolution quadrupole-orbitrap mass spectrometer (Q Exactive™ HF Hybrid Quadrupole-Orbitrap, Thermo Fisher Scientific). An electrospray ionization interface was used as ionization source. Analysis was performed in negative ionization mode. A QC sample was analysed in MS/MS mode for identification of compounds. The UPLC was performed using a slightly modified version of the protocol described by Catalin et al. (UPLC/MS Monitoring of Water-Soluble Vitamin Bs in Cell Culture Media in Minutes, Water Application note 2011, 720004042en). Peak areas were extracted using TraceFinder 4.1 (Thermo Scientific). Identification of MtriDAP-MP were performed using retention time (compared against an authentic standard) and accurate mass (with an accepted deviation of 3 ppm), and for UDP-MtriDAP and UDP-MtetraDAP using accurate mass alone (with an accepted deviation of 3 ppm).

2. Results

2.1. Identification of the F4 Clone

The F4 clone has been identified by screening 5000 metagenomic Escherichia coli clones from a healthy donor library, because this clone demonstrated activity on AP-1 and NE-KB reporter system in HT-29 cells.

Sequencing analysis demonstrated that this clone F4 encodes a contig of about 41 kb coming from a Firmicutes Gram-positive bacterium. By systematically mutating the F4 clone it was shown that the component responsible of said biological activity is a putative MsbA1-MsbA2-like heterodimeric transporter.

The sequences encoding said transporter were identified (SEQ ID NO:3 and SEQ ID NO:4). In the contig, the two genes were found to be consecutive, in the same Open Reading Frame (ORF) (SEQ ID NO:8).

2.2. Subcloning of the Target Genes of Interest

The F4 clone was shown to be very unstable and to easily loose its biologic activity. The sequence of the genes of interest, encoding the MsbA1-MsbA2-like transporter, were therefore subcloned so as to complement the loss of activity of the parental clone. To do that, the sequence encoding the MsbA1-MsbA2-like transporter (SEQ ID NO:8) was amplified from the original F4 fosmid and cloned into the arabinose induced pBAD30 vector (containing the arabinose PBAD promoter of the araBAD “arabinose” operon). The construction was verified by sequencing (see methods for details).

This new construction was called pBAD-MsbA1-A2. It was used to complement the F4D5 transposed clone (containing the inactive msbA1-msbA2-like genes). The complemented and functional clone is called F4D5:MsbA1-A2 clone (also referred as “A1A2”).

The appropriate transformants were selected on LB-agar plates containing Ampicillin (100 μg/ml), Kanamycin (50 μg/ml) and Chloroamphenicol (12.5 μg/ml). Various L-Arabinose percentages were tested, to induce the Ara promoter and to effectively produce active compounds into the liquid bacterial culture. Importantly, complementation of the transposed clone (F4D5) with the construction encoding the MsbA1-MsbA2-like transporter allowed to restore the original activity of the F4 clone, confirming the key role of said genes for the biological activity of these bacteria. For this reason, the complemented F4D5:MsbA1-A2 clone (A1A2) was used for all the experiments described hereafter. The F4D5-pBAD clone (F4D5 clone transformed with an empty vector) was also used as negative control.

2.3. Biological Activity of the Polypeptides Encoded by the Target Genes of Interest

Surprisingly, the clone F4D5:MsbA1-A2 was shown to be able to produce a supernatant displaying anti-inflammatory properties.

2.3.1. This Supernatant Contains Compounds that Enhance the Secretion of IL10 by Dendritic Cells (DCs)

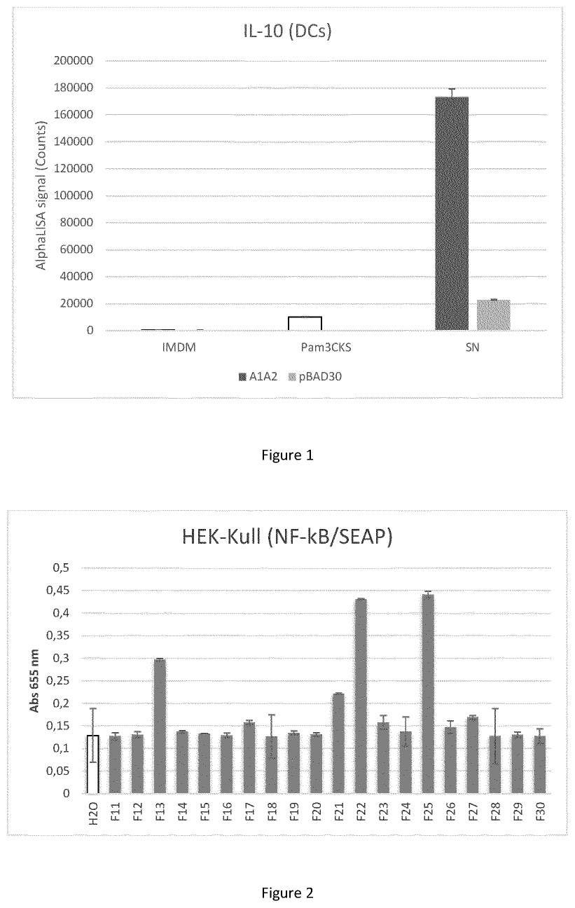

As shown on FIG. 1, the clone F4D5:MsbA1-A2 was shown to increase the secretion of IL10 by DCs.

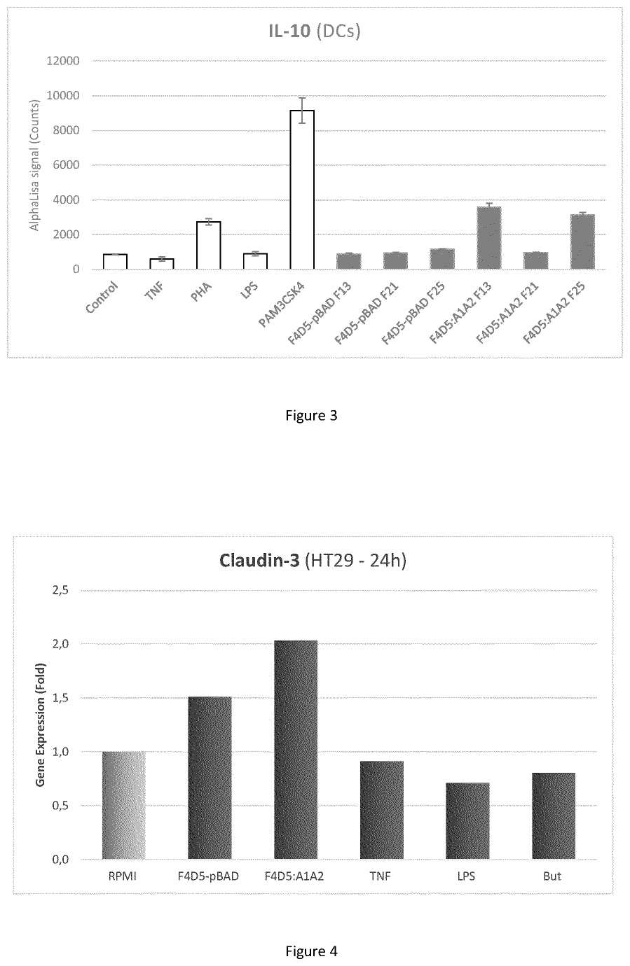

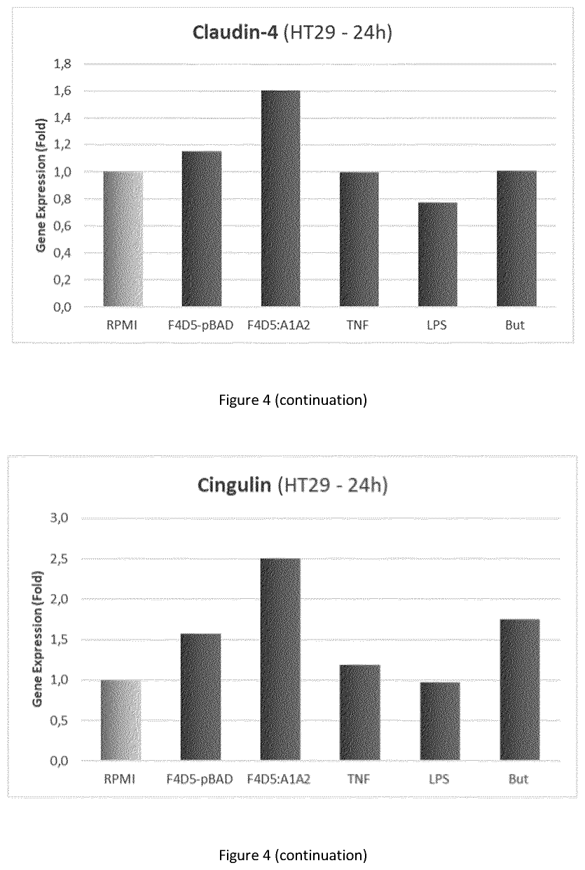

HPLC fractionation of pre-purified supernatant through a Hypercarb column and an Acetonitril-TFA gradient allowed to identify three biologically active fractions (F13, F22 and F25) when tested on HEK Null reporter cells (FIG. 2). These three fractions were then tested on DCs and two of these (F13 and F25) were able to stimulate IL-10 secretion and to induce an upregulation of CD80, CD83, and CD86 differentiation clusters. (FIG. 3).

The bioactive compounds present in these fractions were then characterized.

2.3.2. This Supernatant Contains Bioactive Anti-Inflammatory Muropeptide Precursors.

The purification of the two active fractions (F13 & F25) resulted in the identification of three compounds corresponding to the following muropeptide precursors:

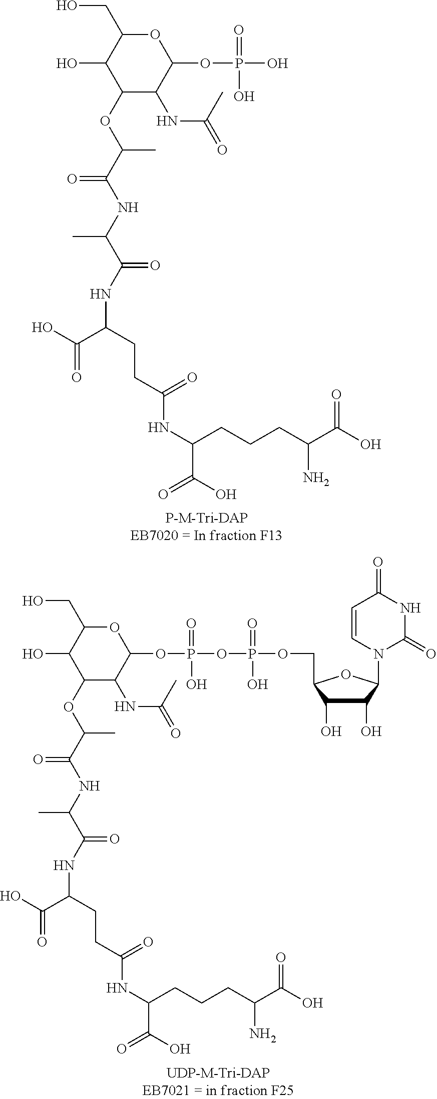

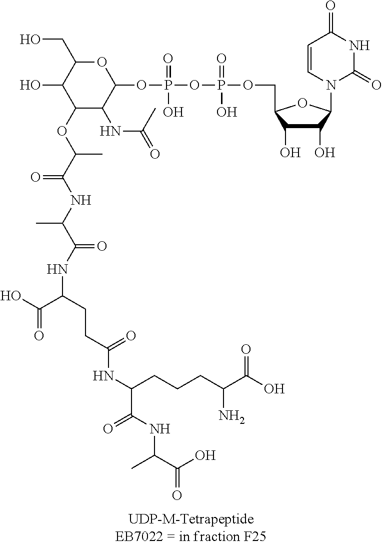

EB7020 is a M-triDAP monophosphate having the formula C₂₆H₄₄N₅O₁₈P. Its exact mass is 745,2419; its Molecular weight is of 745,6240.

EB7021 is a UDP-M-Tri-DAP monophosphate having the formula C₃₅H₅₅N₇O₂₆P₂. Its exact mass is 1051,267; its Molecular weight is of 1051,790.

EB7022 is a UDP-M-tetrapeptide monophosphate having the formula C₃₈H₆₀N₈O₂₇P₂. Its exact mass is 1122,304; its Molecular weight is of 1122,868.

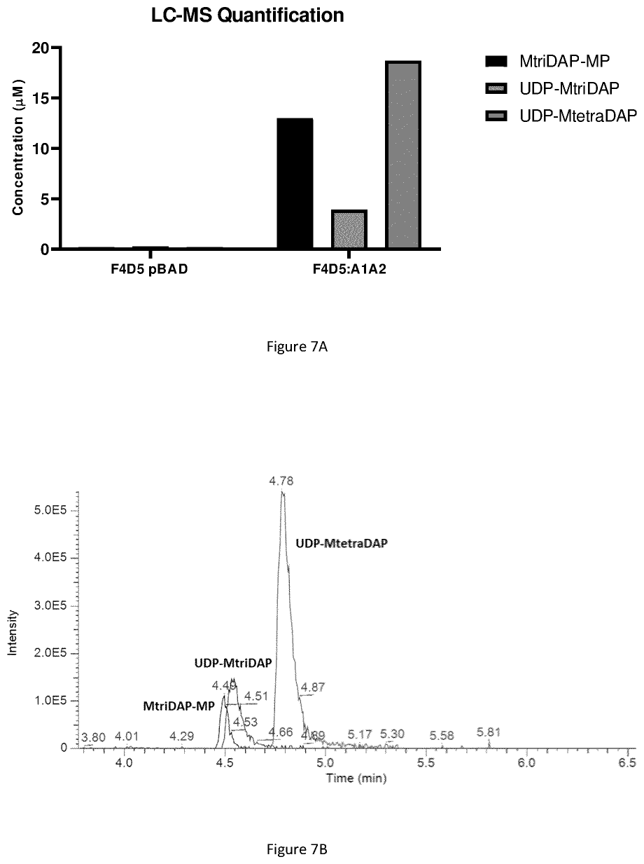

The quantity of the three muropeptides that are secreted has been evaluated by LC-MS (FIG. 7). EPI300 bacteria that are transformed with the vector of the invention encoding the heterodimeric transporter are able to secrete between 5 and 20 μM of each muropeptide precursor (FIG. 7A).

These muropeptide precursors participate in the protective effect of the epithelium which is specifically observed in inflammatory circumstances.

2.3.3. F4D5-MsbA1-A2 Transformed Epi300 Bacteria Secrete these Bioactive Anti-Inflammatory Muropeptide Precursors.

M-TriDAP-MP EB7020 was quantified from F4D5-MsbA1-A2 and F4D5-pBAD supernatants by LC-MS using a calibration curve generated with chemically synthesized M-TriDAP-MP (FIG. 7).

UDP-M-TriDAP EB7021 and UDP-M-TetraDAP EB7022 amounts were extrapolated from their known mass using the same calibration curve.

Very high yield, about 10 μM range for M-Tri-DAP-MP and UDP-M-TetraDAP have also been obtained.

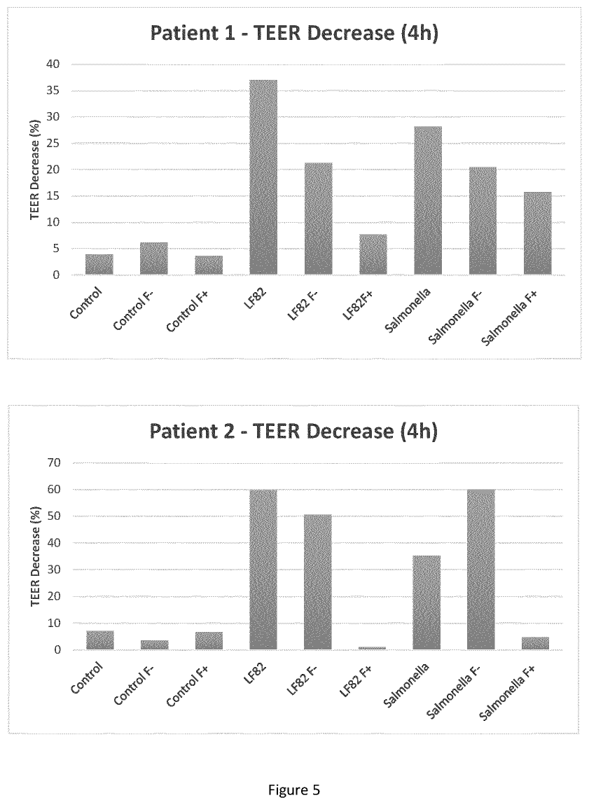

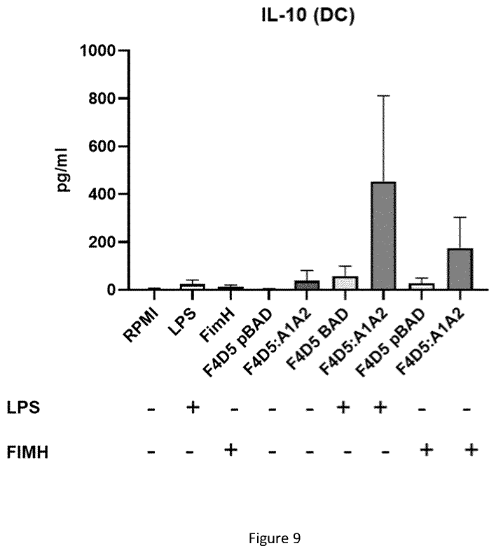

Pre-purified fractions from Epi300 bacteria transformed with F4D5:A1A2, but not those transformed with negative control F4D5-pBAD, are able to enhance the production of IL10, when combined with TLR activators (FIG. 9) and to protect human ileal resections against loss of TEER (FIG. 5).

Many studies were then performed to investigate the anti-inflammatory activity of these muropeptide precursors:
Array on Dendritic Cells stimulated by the IL10 positive fractions of the F4D5-MsbA1-A2 clone
   Evaluation of IL-10 secretion induced by chemically synthesized EB7020 in presence of bacteria supernatant or LPS
RT-Q-PCR on HT-29 epithelial cells
Ex-vivo studies.

Due to the complexity of the purification process and the difficulties to produce sufficient amounts of purified fractions, most of the experiments were realized using samples submitted to several pre-purification steps (ultra-filtration+acetone precipitation+HPLC-HILIC fractionation). Moreover, the EB7020 compound has been successfully synthetically produced.

2.3.4. This supernatant contains compounds that are involved in the maintaining of epithelial integrity.

Transcriptomics was carried out on PBMC derived DCs from 3 donors.

DCs were stimulated (6h) with:
TNFα, PHA, LPS, PAM3CSK4, MDP=Positive control
F13, F25=HPLC fractions obtained from the complemented and functional F4D5-MsbA1-A2 clone
F4D5-pBAD-F13, F4D5-pBAD-F25=HPLC fractions obtained from the F4D5-pBADclone (negative control)

Data analysis was realised using TIBCO Spotfire software. The F4 fractions (from both positive and negative clones) were analysed individually and compared to known agonists (TNFα, PHA, LPS, PAM3CSK4, MDP).

As shown in Table 2, most of the upregulated genes when DCs are contacted with these fractions are involved in the maintaining of epithelial integrity (PERP, PMP22, SLC39A14,) suggesting a protective role of the compounds present in these fractions. This hypothesis is reinforced by the fact that most pro-inflammatory genes (like those encoding IL6, CCL1, CCL8, TNF-α) are downregulated, as shown in Table 3.

TABLE 2
List of Up-regulated genes by F25 fraction.

| GeneSymbol | TNF-α | PHA | LPS | PAM | MDP | F25/pBAD |
|---|---|---|---|---|---|---|
| PERP | 1.0 | 1.7 | 1.8 | 2.6 | 1.6 | 2.0 |
| PMP22 | 0.8 | 0.7 | 0.8 | 1.0 | 1.0 | 2.4 |
| SLC39A14 | 1.2 | 1.0 | 1.0 | 1.6 | 0.9 | 2.3 |

Up-regulated genes (all of them are involved in epithelial integrity). Data of F25 from positive clone are normalized versus the corresponding fraction from the negative clone.

TABLE 3
List of Down-regulated genes by F25 fraction.

| GeneSymbol | TNF-α | PHA | LPS | PAM | MDP | F25/pBAD |
|---|---|---|---|---|---|---|
| IL6 | 32.9 | 525.8 | 756.8 | 718.1 | 9.4 | 0.3 |
| CCL1 | 4.2 | 25.4 | 38.6 | 30.6 | 3.1 | 0.6 |
| CCL8 | 11.2 | 193.6 | 271.6 | 64.8 | 4.1 | 0.5 |
| TNF-α | 5.6 | 17.5 | 22.6 | 28.4 | 3.5 | 0.7 |

Down-regulated genes (pro-inflammatory cytokine and chemokines). Data of F25 are normalized versus F25 pBAD30 values.

Because modulation of genes linked to cellular adhesion (PERP) was observed in DCs, the effect of the bioactive supernatant was investigated on the modulation of several genes regulating tight junctions in the intestinal epithelial cell line HT-29 (see methods for details).

For this experiment pre-purified fractions from positive F4D5-MsbA1-A2 and from F4D5-pBAD negative clones) were used.

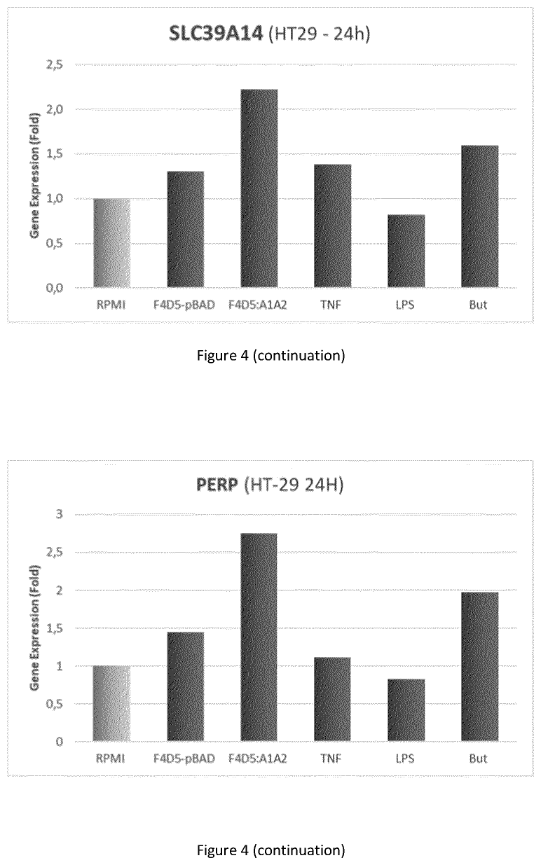

As shown in FIG. 4, the tested fraction of the F4D5-MsbA1-A2 clone is able to upregulate various genes linked to tight junctions in epithelial cells. This means that the bioactive compounds present in the supernatant of this clone have a protective role on the epithelial barrier.

2.3.5. Chemically Synthesized EB7020 Generates IL-10 Secretion from Human DCs.

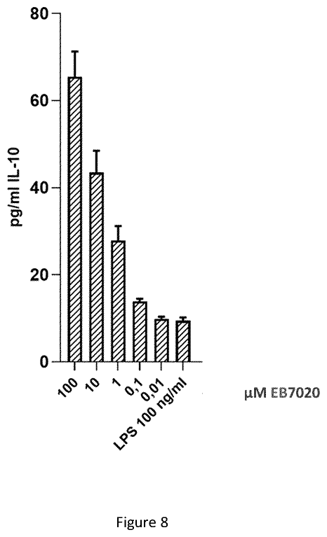

As shown in FIG. 8, IL-10 secretion is induced by chemically synthesized EB7020(F13) in presence of LPS.

2.3.6. Synergy Between the Pre-Purified Supernatants from Transformed Bacteria and TLR Activators.

According to the literature NOD1 and NOD2 agonists synergise with LPS which is a TLR4 ligand.

Pre-purified fractions from both positive (F4D5:MsbA1-A2) and negative (F4D5-pBAD) clones were tested in co-stimulation with either LPS or FimH (a bacterial adhesin) for their capacity to induce IL-10 secretion from DCs. The results are shown in FIG. 9.

It is observed a strong synergy of the fractions of the invention with TLR4 agonists such as LPS or FIMH on IL-10 secretion. This is consistent with literature pertaining to the MoA of muropeptide precursors.

2.3.7. Ex Vivo Experiments.

Ex vivo experiments were also performed to study the properties of the F4 supernatant on a different and more physiological system. The effect of the bioactive compounds was assessed on ileal explants treated with live bacteria (E. coli LF82 and Salmonella thyphimurium) in order to mimic an intestinal inflammation.

Human ileal explants were obtained from two patients and treated within 3 h from the surgery (only the non-inflamed part of the tissue is considered for the experiment). Explants were pre-treated for 1h with the control or active fractions before being loaded with live bacteria. For this experiment, pre-purified samples were used (10 kDa filtered+acetone precipitation+C18 SPE). Infection was done during 4h. For each patient, measurement of transepithelial resistance (TEER), and histology were realized.

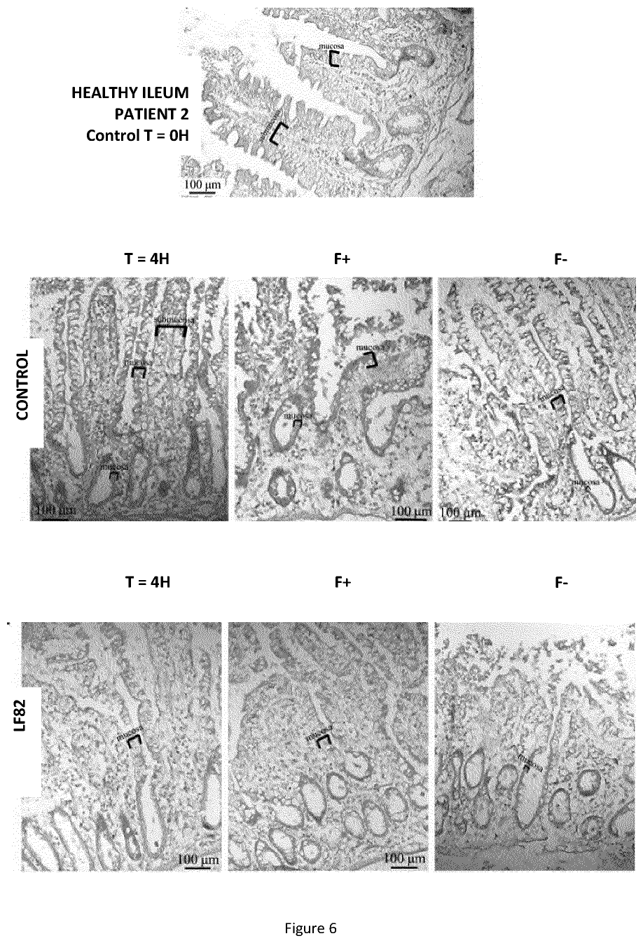

In the following and in FIGS. 5 and 6, the fraction obtained from the active F4D5:MsbA1-A2 clone is indicated as “F+” while the fraction obtained from the negative control (F4D5-pBAD) is indicated as “F−”.

Measurement of TEER was found to be comparable between the two patients. In both cases, the positive sample exerted a protective function on the tissue loaded with the pathogenic bacteria (FIG. 5).

In parallel with the measurement of TEER on ex vivo human explants, a comparative histology study was performed on patient 2, for which the tissue at TO was found non-inflammatory, with a mucosa of normal thickness. The mucosa and the submucosa stand perfectly.

Infection with LF82-GFP Bacteria (FIG. 6).

After 4 hours of incubation with culture media, the thickness remained normal and the mucosa was normal at the level of crypts and on the first third of the villosities. The normal thickness of the mucosa at the level of the crypt showed that the tissue was not inflammatory.

The explants incubated with F+ gave similar structure compared to control at T=4h.

However, after 4 hours of incubation with the LF82-GFP bacteria, a strong release of the submucosa and a strong secretion of mucus were observed, which resulted in a strong desquamation of the mucosa at the level of the villosities. At the level of the crypts, the thickness of the mucosa greatly diminished, probably due to an important inflammatory state.

After 4 hours of incubation with the LF82-GFP bacteria in the presence of the fraction F−, no differences were observed with respect to the bacteria alone. Significant and greater desquamation at the level of the villosities could be due to an irritating effect of the fraction F-which was added to an installed inflammatory state caused by the LF82-GFP bacteria.

On the other hand, the fraction F+ clearly improved the general state of the tissue. It was found that the treated tissue corresponded to the T 4h control without infection. Moreover, the bioactive compounds present in the fraction F+ blocked the inflammatory effect caused by LF82-GFP infection.

Infection with Salmonella typhimurium

As with infection with E. coli LF82-GFP, the infection by Salmonella typhimurium bacteria caused a strong destruction of the mucosa at the level of villosities. The addition of the compound F+ reduced the desquamation to the level of control state, with reappearance of a continuous mucosa around the submucosa even when the submucosa was lost.

In conclusion, a clear protective effect was observed for the bioactive compounds present in F+ on the infection caused by LF82-GFP or Salmonella typhimurium bacteria. Indeed, the bioactive compounds present in F+ strongly reduced the desquamation of the mucosa and therefore the inflammatory state of the tissue caused by the bacterial infection. On the other hand, the compounds present in F− had no effect on bacterial infections, either with LF82-GFP or with Salmonella typhimurium.

1. A recombinant nucleic acid encoding a heterodimeric protein comprising a first polypeptide whose amino acid sequence is SEQ ID NO: 1 or a homologous sequence thereof having the same biological function, and a second polypeptide whose amino acid sequence is SEQ ID NO: 2 or a homologous sequence thereof having the same biological function.

2. The recombinant nucleic acid of claim 1, encoding a heterodimeric protein whose first and second polypeptides have a sequence having at least 80% sequence identity with SEQ ID NO:1 and SEQ ID NO:2 respectively.

3. The recombinant nucleic acid of claim 1, comprising a first polynucleotide having the sequence SEQ ID NO:3 and a second polynucleotide having the sequence SEQ ID NO:4, or homologous sequences thereof, in the same Open Reading Frame.

4. The recombinant nucleic acid of claim 1, comprising the polynucleotide having the sequence SEQ ID NO:8, or an homologous thereof sharing at least 80% identity with said sequence and having the same biological function.

5. The recombinant nucleic acid of claim 1, comprising the polynucleotide having the sequence SEQ ID NO:5, or an homologous thereof sharing at least 80% identity with said sequence and having the same biological function.

6. The recombinant nucleic acid of claim 3, wherein said first and second polynucleotides are operably linked to regulatory sequences allowing their expression in a host cell.

7. An expression vector or cassette comprising the recombinant nucleic acid as defined in claim 1.

8. (canceled)

9. A recombinant host cell, comprising the expression vector or cassette of claim 7.

10. The recombinant host cell of claim 9, comprising a polynucleotide having the sequence SEQ ID NO:5 a polynucleotide having the SEQ ID NO:8.

11. The recombinant host cell of claim 9, wherein it is a genetically engineered prokaryotic bacterium.

12. The recombinant host cell of claim 11, wherein it is a non-pathogenic bacteria chosen from the group consisting of: Bacillus, Bacteroides, Bifidobacterium, Brevibacteria, Clostridium, Enterococcus, Escherichia coli, Lactobacillus, Lactococcus and Saccharomyces, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii.

13. The recombinant host cell of claim 11, wherein it is a Gram-negative non-pathogenic bacterium.

14. (canceled)

15. A pharmaceutical composition comprising the recombinant host cell of claim 11 and a pharmaceutically acceptable carrier.

16. A pharmaceutical composition comprising at least one muropeptide chosen from the group consisting of: 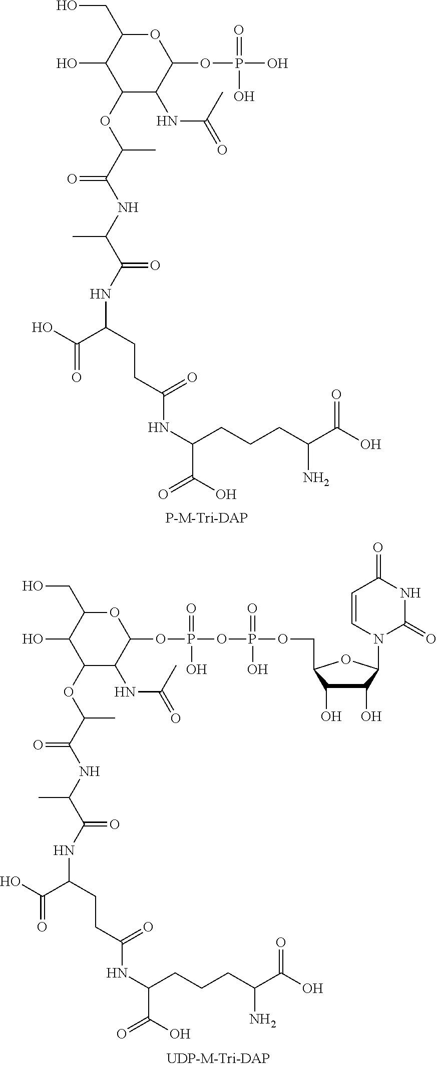 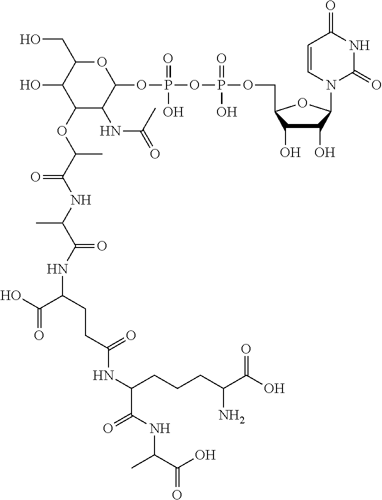
and a pharmaceutically acceptable carrier.

17. A method for:
reducing gastrointestinal inflammation,
reducing intestinal mucosal inflammation,
increasing wound healing,
increasing intestinal epithelial cell proliferation, or for
treating or preventing epithelial barrier function disorders,
said method comprising the step of administering the recombinant host cell of any of claim 8 to a patient in need thereof.

18. The method of claim 17, wherein said epithelial barrier function disorder is chosen in the group consisting of: inflammatory bowel disease, ulcerative colitis, pediatric UC, Crohn's disease, pediatric Crohn's disease, short bowel syndrome, mucositis GI mucositis, oral mucositis, mucositis of the esophagus, stomach, small intestine (duodenum, jejunum, ileum), large intestine (colon), and/or rectum, chemotherapy-induced mucositis, radiation-induced mucositis, necrotizing enterocolitis, pouchitis, a metabolic disease, celiac disease, irritable bowel syndrome, or chemotherapy associated steatohepatitis (CASH).

19. (canceled)

20. A recombinant heterodimeric protein comprising a first polypeptide whose amino acid sequence is SEQ ID NO: 1, and a second polypeptide whose amino acid sequence is SEQ ID NO: 2, or homologous sequences thereof sharing at least 80% identity with said sequences and having the same biological function.

21. The recombinant heterodimeric protein of claim 20, comprising the amino acid sequence of SEQ ID NO:9 or a homologous sequence thereof sharing at least 80% identity with SEQ ID NO:9 and having the same biological function.

22. The recombinant heterodimeric protein of claim 20, wherein said biological function is measured by analysing the presence of the muropeptides as defined in claim 16 in the supernatant of cells overexpressing said recombinant heterodimeric protein.

23. The recombinant heterodimeric protein of claim 20, wherein said biological function is measured by contacting the supernatant of cells overexpressing said heterodimeric protein with dendritic cells, and observing the upregulation of IL-genes in said cells, or the secretion of IL-10 by said cells.


SEQUENCE LISTING (9 sequences)

SEQ ID NO: 1 - protein, 588 aa, Unknown
  misc_feature: Gram-positive bacterium from the phylum Firmicutes
MFQLKWVWKQMEGFRKRYIFALFSTALLAMLTLGNSVITASIMDTVFQPLTESGVVTQQVVHHLAVLVAVLIGFTLFRTSFQYLSIMTYEGCSQKLIFKLRRDLYKNMQEQDQDFFSKTRTGDLMTRLTGDLDMVRFAVAWVVRQLIHCTVLFVTTSIVFLVTDWLFALSMLAVTPIIFALTLAFSKRVHPLYVDLRERLSLLNTQAQENISGNRVVKAFAREDYEIDRFDEKNADYKKANTRASLLWLQYSPYIEGLSQSLSIAVLLVGGVFLITGRISIGTFTLFNGLTWTLTDPMRMLGMHLNDLQRFFASSNKIIELYYAKSTITSRPDAKKVDTRLKGEIDFSGVDLNLHGQPVLRHIDLHINPGETVAIMGPTGSGKTSLVNLIPRFTDVSGGTLTIDGTPVGRYDLQGLRHAIGIATQDVFLFSDTVDGNIAYGDSSLSEDDVKRYAKMADVDFVEKLPDGFDTLIGERGTGLSGGQKQRIALARALAVRPSILILDDTTSAVDLETEKYIQEQLASLDFPCTKIIVAQRISTTKRADKIVILDKGRVVDIGTHEELSQRPGYYREVFLLQNGMEEEKEVV

SEQ ID NO: 2 - protein, 589 aa, Unknown
  misc_feature: Gram-positive bacterium from the phylum Firmicutes
ARNKFDVDETLETPFNIKHLLRAGVYIGRHKKKMILSLLFSAISAACSLLGPMLIQRAIDVSVPQKDYTELMVVLAVIMLVSIVASVLFARARSKYMIVVGQEIIYDIRKDLFEHLQKLPFQFYDDRPHGKILTRVINYVNSVSDALSNGIINFVLEIFNLILIAVFMFLCDVRLSLIVMAGIPLFLVIVLLIKPAQRRAWQDVSNKSSNINAYLHESLDGMKITQAFTREEENRGIYEKLNKKCYQTWMKAQYTSNLVWYSVDNISTWVVGAMYLIGLWMLGPAMQIGTLIAISSYAWRFWQPILNLSNLYNTFINAVAYLERIFEMIDEPVTVDDAPGATELPPITGRVTFDDVTFSYDGQINILEHFNLDVKPGESIALVGPTGAGKTTVVNLISRFYNIDRGRLLLDGHDIAQVTLRSLRSQMGIMLQDSFIFSGTIMDNIRYGRLDATDEEVIAAAKTVRADEFIREMEDGYYTQVNERGSRLSQGQRQLVAFARTLLSDPKILVLDEATSSIDAKTERLVQEGLNALLKGRTSFIIAHRLSTIKNCDRILYISNKGIAEMGTHQQLLEKKGYYYHLYTAQLES

SEQ ID NO: 3 - DNA, 1767 bp, Unknown
  misc_feature: Gram-positive bacterium from the phylum Firmicutes
atgtttcagttaaagtgggtgtggaagcagatggagggcttccgaaagcggtacatctttgccctgttctccacggccctgctggcgatgctgaccctgggcaactcagtcatcaccgccagcattatggacacggtgttccagccgcttaccgaaagcggtgtggtcacccagcaggtggtgcaccatctggcggttctggttgcggtgctcattgggtttacgttgttccgcaccagctttcagtacctatccattatgacctatgagggctgttcccaaaagctaatcttcaagctccgccgggatttgtacaagaatatgcaggagcaggaccaggacttcttctccaaaacccgcaccggcgacctgatgacccgcctcaccggcgacctggatatggtccggtttgccgtggcctgggtggtgcggcagctgatccactgcacagtgctgtttgtcaccacctccattgtgtttttggtgacagactggctctttgccctgtccatgctggcggtaacgcccattatctttgccctgacgctggccttctccaagcgggtgcaccccctgtatgtggacctgcgggagcgcctctctttgctaaacacccaggcccaggagaacatctctggcaaccgggtggtaaaggccttcgcccgggaggactatgagatcgaccggttcgatgagaaaaatgccgactataaaaaggccaacaccagggcctccctgctgtggctgcagtacagcccctatattgagggcctgtcccagtccctctccattgcggtgctgctggtggggggcgtgttcctcatcaccgggcgcatctcaattgggaccttcaccctgttcaacggcttgacctggacgctgaccgaccccatgcgcatgttgggcatgcacctcaacgacctgcagcgtttcttcgccagttccaataagattatcgagctgtactatgccaagtccaccatcacttcccggcccgacgccaaaaaggtggacacccgcctgaagggggagatcgacttctccggcgtggacctgaacctccacggccaaccggtgctgcgccacattgacctgcatatcaatcctggcgaaacggtggccattatggggcccaccggctctggaaagacctcgctggtgaatttgatcccccggtttacggacgtcagcggcggcaccctcaccatagacggcacgccggtggggcgctatgacctgcagggcctgcgccacgctatcggtatcgccacccaggacgtgttcctgttttccgacacggtggacggcaatatcgcctatggcgactcctccctctccgaggatgacgtgaagcgctatgccaaaatggcggatgtggactttgtggaaaagcttcccgatggcttcgacaccctcattggcgagcggggcaccggcctttccggcggccagaagcagcgcatcgccctggcccgggccctggccgtgcggccctccatcctcatcttggatgacaccaccagcgccgtggacctggaaaccgagaagtacatccaagagcagctggccagcttggacttcccctgcaccaagatcatcgtggcccagcgcatttccaccaccaagcgggcggataagatcgtcattttggataagggccgggtagtggacatcggcacccacgaggagctgtcccagcggcccggctactaccgggaagtgttcctgctgcaaaacggcatggaagaggaaaaggaggtggtttaa

SEQ ID NO: 4 - DNA, 1770 bp, Unknown
  misc_feature: Gram-positive bacterium from the phylum Firmicutes
atggcacgcaacaagtttgacgtggacgaaaccctggaaaccccatttaacataaagcatctgctccgggccggcgtgtacattggccgccacaagaaaaagatgattttgtccctgctgttctccgccatttccgccgcctgctctctgctggggcccatgctcatccagcgggccattgacgtgtccgtgccccaaaaggactacaccgagctggtggtgctggccgtcatcatgctggtgtccattgtggcctctgtgctgttcgcccgggcccgctccaagtacatgattgtggtgggccaggagattatctacgacatccgcaaggacttgttcgagcacctgcagaagctgcccttccagttttacgatgaccggccccacggcaagattttgacccgcgttatcaactatgtcaactccgtgtcggatgccctctccaacggcatcatcaactttgtgctggagatcttcaacctgatcttgattgccgtgttcatgttcctgtgcgatgtgcgcctgagcctgatcgtcatggcgggcatccccctgtttctggtcatcgtgctgctcatcaagcccgcccagcgccgggcctggcaggatgtgtccaacaagagctccaatatcaacgcctacctccacgaaagcctggacggcatgaagatcacccaggccttcacccgggaggaggagaaccgcggcatctacgagaagctgaacaagaagtgctatcagacctggatgaaggctcagtacacctccaacctggtgtggtactctgtggacaacatctccacctgggtggtgggcgccatgtacctcatcggcctgtggatgctggggcccgccatgcagattggcaccctcattgccatttcctcctacgcttggcggttctggcagcccattttgaacctgtccaacctgtacaacaccttcatcaacgcggtggcctatctggagcgcatcttcgagatgattgacgagcccgttactgtggacgatgcccccggcgccaccgagctgccccccatcaccggccgtgtcaccttcgatgacgtgaccttctcctatgatgggcaaatcaacatcttagagcacttcaacctggatgtaaagcccggcgagtccattgccctggtgggccccaccggcgccggcaagaccactgtagtgaacttgatctcccggttctataacatcgacaggggacgcctgctgctggatggccacgacatcgcccaggtgaccctccgctcccttcgctctcaaatgggcattatgcttcaggacagcttcatcttctccggcaccattatggacaacatccgctatggccgcctggacgccaccgatgaggaggtcatcgccgcggccaagaccgtgcgcgcggatgagttcatccgggaaatggaggatggctattacacccaggtcaacgagcggggctcccgcctgtcccagggccagcggcagctggtggcctttgcccgcaccctgctctccgaccccaagattctggtgctggatgaggccacctcttccatcgacgccaaaaccgagcgcctggtgcaagagggcctcaacgcccttctgaaaggccgtaccagctttatcatcgcccaccgcctttccaccatcaagaactgcgaccgcatcctgtacatttccaacaagggcattgcggagatgggcacccatcagcagctgttggagaagaagggatattattaccatctgtacacggcccagctggagagctga

SEQ ID NO: 5 - DNA, 32767 bp, Unknown
  misc_feature: Gram-positive bacterium from the phylum Firmicutes
  misc_feature (10000)..(10000): n is a, c, g, or t
  misc_feature (17318)..(17318): n is a, c, g, or t
  misc_feature (18085)..(18085): n is a, c, g, or t
  misc_feature (23533)..(23533): n is a, c, g, or t
  misc_feature (23734)..(23734): n is a, c, g, or t
  misc_feature (31312)..(31312): n is a, c, g, or t
tcggtacccggggatcccacgtacaacgacacctagaccactttggaaggcgcgggctatttcccgcgcagctcgcggatcaggtaatccacctgatccaaagcctgctgcttttgatggatctcctccaacaggcagcagcggcaccgccgcagcagccgaagccgctccccgactgtatccgatccctgttccgccagcctgacaaagtgcttcagcgtgcccagttccatcccggcttgctgcaaaaaccgaaacgaaactgcccgctgcagcgcttcctccccataatgccgggtaccgtccggcgcctgcaccccttgcaggagcccattttcctcatacagcctcagcagttccggcggcagatgaaatcttctgcccgcttctaacaggtccatgtgtctccccccgtttctccccaaaagggtcttgtgttgggaagtgcccttccactctcattataggggacgggaagccgcctgtctaatacccgtttttcataaggcgcaatgcttacagggcataaaaagcgctcaaccggctggacggagggtttccttggcaaactgaatccacttggagaccgccgggctgaagggctgcttgcgcttccaggccaggatgctggtggccttcagctctggatacagggggcggcaggtgattttttccgggtcccagaaggagagggagccccggatcgtaatggaataggccagctggttatgggccacaatggaactgatggccggcagattggaggtaaagaccacattcagcttgtcaaactcctctccaaaccagcctgccagctcactttgcacactgagcctgcggggaagaattaaaggctggccggccaaatcctgaggagtcacccggtccaaccgggccaagggggcgtcagggtgcatcaccgcccaccactgctcctgccgggccagccggatgtattcatatttttctacgctcaccggctccatcagcaggccaatatccgtcaggccccggtccatcctgtcctggatgtggtcggcagtaccggtgtacaggtcaaagcgcaccgcgggatagcgcgcgcggaacgcccggaacagcggcggcagcagctccacagaggccaggtctccgcagcccagggaaacagtcccctcaatccgctcgtcctgctccgtcagttccttctcggtcttatccaccagctccagaatctcctctgcccggcggcgcagcagcaggccctcattggtaagggcaatcttgcgggcgccccggtcaaacagtttgacccccagttcctcctccatctggccgatctgccggcttaaagtaggctgggtgaggtgaagcagctcggctgccttggtgatgctctcctcccgtgctaccgccaggaaataccgcaaaacccgaatttccacaggcctttccccctttctttttccccatcatagcagactcatgctcaaaaagcaatctcttttcctttcatgcttcaatggcatttcaagcaggcagccttctggaggaccgcctgctttttccctgtgaaaaaaagcaagcgcgaaccctgccgccgctctggcgccgtgggctgcgcgcttgcttttgcgctctattccgttatcccagagggagctctctccctgttcagctctccagctgggccgtgtacagatggtaataatatcccttcttctccaacagctgctgatgggtgcccatctccgcaatgcccttgttggaaatgtacaggatgcggtcgcagttcttgatggtggaaaggcggtgggcgatgataaagctggtacggcctttcagaagggcgttgaggccctcttgcaccaggcgctcggttttggcgtcgatggaagaggtggcctcatccagcaccagaatcttggggtcggagagcagggtgcgggcaaaggccaccagctgccgctggccctgggacaggcgggagccccgctcgttgacctgggtgtaatagccatcctccatttcccggatgaactcatccgcgcgcacggtcttggccgcggcgatgacctcctcatcggtggcgtccaggcggccatagcggatgttgtccataatggtgccggagaagatgaagctgtcctgaagcataatgcccatttgagagcgaagggagcggagggtcacctgggcgatgtcgtggccatccagcagcaggcgtcccctgtcgatgttatagaaccgggagatcaagttcactacagtggtcttgccggcgccggtggggcccaccagggcaatggactcgccgggctttacatccaggttgaagtgctctaagatgttgatttgcccatcataggagaaggtcacgtcatcgaaggtgacacggccggtgatggggggcagctcggtggcgccgggggcatcgtccacagtaacgggctcgtcaatcatctcgaagatgcgctccagataggccaccgcgttgatgaaggtgttgtacaggttggacaggttcaaaatgggctgccagaaccgccaagcgtaggaggaaatggcaatgagggtgccaatctgcatggcgggccccagcatccacaggccgatgaggtacatggcgcccaccacccaggtggagatgttgtccacagagtaccacaccaggttggaggtgtactgagccttcatccaggtctgatagcacttcttgttcagcttctcgtagatgccgcggttctcctcctcccgggtgaaggcctgggtgatcttcatgccgtccaggctttcgtggaggtaggcgttgatattggagctcttgttggacacatcctgccaggcccggcgctgggcgggcttgatgagcagcacgatgaccagaaacagggggatgcccgccatgacgatcaggctcaggcgcacatcgcacaggaacatgaacacggcaatcaagatcaggttgaagatctccagcacaaagttgatgatgccgttggagagggcatccgacacggagttgacatagttgataacgcgggtcaaaatcttgccgtggggccggtcatcgtaaaactggaagggcagcttctgcaggtgctcgaacaagtccttgcggatgtcgtagataatctcctggcccaccacaatcatgtacttggagcgggcccgggcgaacagcacagaggccacaatggacaccagcatgatgacggccagcaccaccagctcggtgtagtccttttggggcacggacacgtcaatggcccgctggatgagcatgggccccagcagagagcaggcggcggaaatggcggagaacagcagggacaaaatcatctttttcttgtggcggccaatgtacacgccggcccggagcagatgctttatgttaaatggggtttccagggtttcgtccacgtcaaacttgttgcgtgccatttaaaccacctccttttcctcttccatgccgttttgcagcaggaacacttcccggtagtagccgggccgctgggacagctcctcgtgggtgccgatgtccactacccggcccttatccaaaatgacgatcttatccgcccgcttggtggtggaaatgcgctgggccacgatgatcttggtgcaggggaagtccaagctggccagctgctcttggatgtacttctcggtttccaggtccacggcgctggtggtgtcatccaagatgaggatggagggccgcacggccagggcccgggccagggcgatgcgctgcttctggccgccggaaaggccggtgccccgctcgccaatgagggtgtcgaagccatcgggaagcttttccacaaagtccacatccgccattttggcatagcgcttcacgtcatcctcggagagggaggagtcgccataggcgatattgccgtccaccgtgtcggaaaacaggaacacgtcctgggtggcgataccgatagcgtggcgcaggccctgcaggtcatagcgccccaccggcgtgccgtctatggtgagggtgccgccgctgacgtccgtaaaccgggggatcaaattcaccagcgaggtctttccagagccggtgggccccataatggccaccgtttcgccaggattgatatgcaggtcaatgtggcgcagcaccggttggccgtggaggttcaggtccacgccggagaagtcgatctcccccttcaggcgggtgtccacctttttggcgtcgggccgggaagtgatggtggacttggcatagtacagctcgataatcttattggaactggcgaagaaacgctgcaggtcgttgaggtgcatgcccaacatgcgcatggggtcggtcagcgtccaggtcaagccgttgaacagggtgaaggtcccaattgagatgcgcccggtgatgaggaacacgccccccaccagcagcaccgcaatggagagggactgggacaggccctcaatataggggctgtactgcagccacagcagggaggccctggtgttggcctttttatagtcggcatttttctcatcgaaccggtcgatctcatagtcctcccgggcgaaggcctttaccacccggttgccagagatgttctcctgggcctgggtgtttagcaaagagaggcgctcccgcaggtccacatacagggggtgcacccgcttggagaaggccagcgtcagggcaaagataatgggcgttaccgccagcatggacagggcaaagagccagtctgtcaccaaaaacacaatggaggtggtgacaaacagcactgtgcagtggatcagctgccgcaccacccaggccacggcaaaccggaccatatccaggtcgccggtgaggcgggtcatcaggtcgccggtgcgggttttggagaagaagtcctggtcctgctcctgcatattcttgtacaaatcccggcggagcttgaagattagcttttgggaacagccctcataggtcataatggataggtactgaaagctggtgcggaacaacgtaaacccaatgagcaccgcaaccagaaccgccagatggtgcaccacctgctgggtgaccacaccgctttcggtaagcggctggaacaccgtgtccataatgctggcggtgatgactgagttgcccagggtcagcatcgccagcagggccgtggagaacagggcaaagatgtaccgctttcggaagccctccatctgcttccacacccactttaactgaaacatacaattcctcccttttcgcgccgggcagcccggcgtgcgtcatgtctcctgaaaaggcgaaaacctggcctttcaggccggcttcgggccctttcccctctccggaaagggctcgattccaagctcccataattatagccctcgctccaaaaaagggaaggggtttttccaaaattccccttcggtatttcgctgaaattccagaggaaaattttcctttttgcacaggagccggacaatccctttcccgcgttccccagccccggaaaacagcgggctgcgaacccctttaaagggcgccttgcccggcgcaagtcatgacccccggcaaagccaggggcttgggtaatccctagaagggcatcgtaccggcaagcctctcaaggggcactgaaaaatccgccacttgcatcacttgccccacagcctgtaaacgggcgttgtttatccaggatcaacttctttacaatagcaagttgttgctcgcttctcgcttcgctcgatgcttgaagctaaagcttttttactctccccagcagagctgggggttctcatttcgctaaagcatacaaaataaaaatcccccggcgaagccgggggtatttcatagacgggcaaagccctctgttactagcggcacgcgcacagcgcgttggtgcggtctgccagccgcgatcctgttacttgccgcccgtaaacgggctttcattgtttagatgtttcccattgtcatctgctccgccatctggtcttcatccagctggtgtttgatgtactccgcaatccgtttcgtgttcttccctgccgtgtctacgtagtacccccggcaccagaactctcggtttcggtatttatacttcagctccgggaacctttcgtacagcattaggctgctcttccctttcaggaatcccacgaaacttgaaacggagtatttcggcggtatctccaagagcatatgcacatggtccgggcacacttctgcctctactatttttatctttttccactcgcacagctctctcaggatctttcccacttcccgtcgtttctctttgtaaaatacctttctccggtattttggcgcaaatgctatagtaatctaaatcacacattggcggcatatacgacaccaccggcaacaggggttcttcctctatgtacccggctgcgccgagtacgtcggaataatcctccgtggcgcagcctttgatatatacctggcatttctgtttggtgccatcagtagtaatataaattcggtcaacaatcgaatgaactaaagtaaacagagcttcaggctgtgcgtcctttgcaaactcctcaaaggacagcagcctttttttcatgccattcagttcatggacgagatactgattttcagattgagtatcctccattcggcgcagcgcagcttcgcactttgccagctcgtccgtcaattcctttatatcatcctggatgaagcgtttaagggcgtcatctgcctcacgcagatttcgtacctgtgcggcaatatccgcatttaagcgttcaatagcctttttggtttcctggtactctttctcgctttggtcagtacggatcatgctggcaaccctgttctccaaaagttgccggtaatagtcggaatgttcttcttgcaggctggagagtgagtggataacaaattcatcaagcgttacaccctcaacccctttgaaggtacattcctttgccctcaccccagggcaggcatacttgaagcggggcttgccattcgtccagcgattggattctggcagtacacgcagccgttttccgcaaatagggcaatacattaagccggacaacaaagcattggtttttctatggggccggttatacttttctgcaatctcatccagcatattttgtgtttccacccatttccgggagctgataaatccctcatggcgacccacagaaacaatccattcactaatgggtttcctggtaagaagctgtgagaatttgggattgaaaaaggtggaatcctcatcctcaactttcatctggtcggtcttattataagcagtaagcccatgctgaccatcaaagtcggacggttctccaaacaggttcccgtcatgctccagaaaatagttgtaggaatattcatctgccacgcagtagattggattccgcagaatcagccggatggtggatgtggtaaacttcgcgccatgcttcgtttcatatttctcactgatgatatttgccgtctgcaagagagaacgtgtctcccagaaggtgctgtagatttccaatacaatctctttttcttcctgcacagggacgagaaagctaatggcactcttattttttccacttcccgttgtcacacgttgggaagaaaagccggtcggtgttttgcctcccagccagcgcccatcttttgccagctccatcaagttatcctggatacgttcagttagaatatcgcgttcaaattctgcgataatggccagcacctggatgatgattttggatgtgctgatccgcgtattgatattgttggagcagaccaaaagcgtcacatcgtgacgctcaaagtattccagaagccgcatgaggtcggaagttttacggctgattcggtctaatttgtaacaggccacagccttgattttccccatctcaatgtcccggagtagcctttgaaaatctgggcggtcggaatagtaaccacttaagcctttgtcctcataccgggcaaattcgtaatcttccgatagagaaagctggttgatagcatagtcggcactttgcttaaactgaacaccaatgctatctcccttatttgtaatgcgggattttctcgcatatatggccaccaatctatcatcaattcccatctggattcttgctttgcgtttcatattcaaatatcctcctttccaggacggtgagaaaaccgcccttttctccattattataggaacgtcctcatttttgctcaaaggtgcgattatcttctttcaatgtagaaagcaatgcttttaattcttctgcgctaaagtccctcaattcacgaacgaggaactcggcgcgtttttcagcgaactggcgcagggattgctcgctgtctttcccggtatggaatacgatctccataggctgccccctactcattggtgcctcttgaccacttcatagtcaaaaccgggccgctggccgcagtaagtgcagatgtccttttccacccggccagggtgaaccctgcgcaaataataagagcctgtgccgtagaagttgttggcacaggccggacaaaggcacatgattaaatttgggggtgtttcttcgatcaatcccacgctgacagccagggcgcggttgagccggcgaatatgctgttcctccagatgtccgatgtacgtttccagccgccttttatcaatggtgcggagctgctccatcagcaccagagaaggcagctccaggccgttctctgctttgagaaggtaatgggtaggcagcttggccttggcgtccactttgctggatatggctgccacaatgaccgtggggctgtgcttgtttccagtatcattctgaataatcaggaccgggcgataaccttcctgctcggagccgattccccggcccaaatcggcatagtacatatcgccgcgatgatatgttctgttcatgctttatgacctccctttgaaatgcaggcggctgctgccgcctatgtagaaccggacagcagaacagaatgaagaccggggagagataaaacaatcctcttgttttcattggcccgtcctgctgtacggctcatatttcaaagcgttcctatttgtcctcgacaccccggaacactgcgggaagtcatcaaacggctggcagctcaccagctccacggggaatctcacccctcgcgtggttctcacgcaaccgcccccattgcctgcgacggatcacggcagcaaaccgcttcaacgctcggacgttaggctggacgggagtatcgctccgctgctggtcatggccgtaccgggagccgcccggtactggcagcctgctgtttctcgctccttgctgggaagaacaaaccaaagacctacaatttcttttttcggtggtgtctcgttgttttctcccggcagatcaccgcgcagccgctacctgggctttggctttcgccaaaacagcgggaatgtgcttgatgaatgggtatgcagttttcaaggtgccgtgaggccataaaaagaacctctcaccctatttgccgttgagggtgagaggttgcacaacgattattccaaaatttttttaagttttttgagggccttcattttccgaacatgaacagcattgcggtacacgccaagctcgtctgcatattgccgttctgtcttttcctcaaagaacagcgcttggatcagcgcacgttcttctggggtaagcagggaaagcgcggtatgtagcctgtccacttccagcttgtgaagcaccacgctctccacgctttcctgctcatcagagaattgctgggcattttcatccaccagccgatccaaagaatcttcgcggcttgggatgatctgcaccacttggccctcctttccgagaacgaaccgctcggttttcaaatcgttctcaaaatagcgcattttccggtcgccctgcatataggcccggtacacttcatccgtaacctcaacatattggccgcgaatccatattctttttttatcagcgtccacgctgttttacctccgatcagtctgaattgcgagaaattcagactggtcgggcggccccctacaacgtgggaatnggggttcctctatattgaaaaacgcacctgcagaatgggacaacagccccaacagcaagtgcgtttccttttagttcgtgtaaatcatggcatgatggttcagtatgaaaggcgtaaagccacggtgaacagcaagcgattttttgacagcctgcgctctgtcgcattatgtcgaaacgctttttcctccatggcggctccctccgtaagccgccgatcccgccagcgtcagggcgtcgtcagtatgagggcataaagaacggcggccttgattttctcaaagccgccgaaaaagtaatatgggcgtgtcaataagtctgctgcacgacggctttttttcttttgccatcgtccggcagataatgaaaatattatgttttaggttcaggatttactaatgcctttactttcgcaatgaaatcacagtcggttttgcagcagaaaacgccgccataaaattttgtctgttgttcaatcaggaaatcatcgtcgccagacataagcaggaaagggacagtgatccctttttgacgaatttcttcgagcaaatccagtcctgtgccatcccgcatattataatcagaacagattgcttcgactttttgatgttccagcaatcgcaacgcatcttttacccctgttgctgtataaacctcaaaatatttttctaaagcagaggtcaaaaaacctaaaaaacccaaattgtcgtctatgacaagcaattttctcattaacttcccccttctatcagctccttattatgttcttatctgattggtggttagaaaatcagaattattttactttcctgttcagccctttcagaatgaacagcgccagaacagcacaggccagcagaactatgataccaattaagacctgaaccatgcttgcatcgtaatagccggagatattgaaaacggccgtcatgggatataccgtctttagctgttcggacatacttgcgaacagcccagcaaacgaataaatttctgcaaataccagcgccagccagtaaccttttttcatccgggccaccaaggcgataatcggagaaatggcaaagaacacgccgatcccgtccagaagatatgttttcaggtttcccaaaacaaggccaattgaaagagcttcaaagtgcaaaacggcctcaacgagaaaggtaatcgcaaaaagcagaagatagattacgacgctgaacaccagagcaagcaccattttggagagcgtcagcttcacttcatccacgggaatgagccgcagggatttcagggtgtcctcctgttcctcccggcagatcatatagcttcccagcagggcaatgaccgcaggcagcacataaaaggtggcaagggattgggccgctgtcataaaccatccggcaccgtcaatatagcggctttcatagaacgtgaactgaccctgtgcaaagacaatcaacgccaccatgaccgttgcaaaaccggcaatccacaagattttggaacggcggagtttccggcgctccgcccaaagtaaaagttccatttcaaatgcacctcactttctctttttcaaagcagccccagccagcaggacagaagcaacgccccacacgcctatgcagaggaatgcccccggaatattgagtggctgggtaattacgacacccggaatatcccgcataacaatggcggtcatgctggacagcggatgcaggtacatattcaccatcaagaggataaagccgaggaatgtataaaccagcgtcaggcagacgggaagaatataccccttctgcgctgcggcagccgccagtattggcagcaccgcaaaggcggtcagcagagagatttccatacacttccgcagaagataaaggacgctcccgctgtcaaaggcaatatagccgggcaggacgccggtaaggatggacgcggctgcggtaatcagcatgaagcagaccgagtacaccagcaccacagcaaatttgctgaaaaagtacgccatcttattgaccggcacaatccagagctgtttgagcatatcatattggttttcgttatacatcagcatggtacacagcatccccagcaccaccggcaggataatccacggggtatagctgaacgccgtccacttgtaaaactcgatgggttccacgccggttcccttaacgcttgcgaagtagaacacggcggccagcggcataaacagagctgccagcagcatgagccagataaactttctccggcgcagctttagaaattcaatctgtatcagtttaagcaatgccctctccccccgtaatctgcttgaaatagtcctcaagggtatcattgcagatttgggagctgataacagccacatcctgcaccacaagggccttgttgattgccgccatatccagtgcggtgtcatagagtcgcaggttatgttcgtcctgcaccgcataatccgtcacatggaactgccgttccaaaatcagtgaggctttggggacatccgacacctgaagctggatgtatttccgatttttcttttccaaatcctccatgctgctttcttccagcagtacgccgtggtcgatgatgccaatatcatccgccagaagtgcgatttcggaaagaatgtggctggaaatcaggatggttttgccgcgatccacgcttaaatccttgataaaattccgcacttcggcaatgccgatggggtcaagcccgttggtcggttcatccaggatcaaaagctccgggtcatgcaggatggcgttggcaatgccgagacgctgcttcataccgagagaatacttgctgaacagctttttgtcccggtaaggaagccccacgacttccagtgcgttcttgacggcgttgggccggggcgtcccgcgtagcttcgcaaagatttcaaggttttctgtgccggtcaggttcgggtaaaagccgggagtttcgatgatagccccaatgcggggataaatgcgcttctcatggcctttgatgttctggccgaacacatccacttcgccggaagtgattggcgtaaggcccaaaatcattttcataatggtggtcttaccggctccatttcggcccaaaagcccgtaaatccggcccggcttcacatgaatatccacagagctgacggcggtttgctcgccgtaaatcttcgtcagctttttggtttcaatcacaaagtcactcataaagatgcctctctttcttattctgcttcatttttccaaatgacatagttgtaaaggctgccatccggggaatagttgatgaacactttgaaatcgcccttttcacagggaatgacaaattcaacacggatttggtcggagtgttccggcacataacaggacacctccggggaagcgttctccggcagcccgcccagcttatcggcttcttccttccatgtttcctgtatcgcctccgcagaagtattgtcctgtacgccctgcggcagcgcattgaagaaagtttcaaaatctccatccatcatctggttaaagtattcggtagcttcctctgtaatggcctgctggtcgggggcttcgctggaagatggggcggcctgctggcaggcgcaaagcgaaaaagcgaacagaacacagaggaataaactaatcaatcgtttcataggtctttcctttcaataagattgcttttttagagccacaacggatgcggcggcaaatgcggcaccgatcagaagcagcgaacctgcacagacaagcggcgaagtatggggcaaaacgccttgcatcaggctttctaccatagctgcggctgcttcggaaatatggggataaatccccatcacgcttgccagcgggtgtattcccgtaagataggcaggggcaatgactacgggaaccagataaagcagcgttgcgccaataggcaggatatatccttttgacagcgtggcaagaaacacaatcggaagcatggctatggggatcagaagcccgcccgtcagatacaaaaggccagcaaccattagtgtttgggcattcagatcgggaaagcccccagcaatcagcccgccgacaacacataaaagaaaggtaatcaggcacagtcccacggacatcaggatcaccacagcaacttttgaaaagtatagctgcgcttttgtgataggaatgatgagcagttctttcagcgtgtcgttcttgtgttcgtcaaaaaataaggttgtggcgaacattcccagcacaatcggcaggaacaggctggacaggttcttaaacgccagtgtgtagaggtcgccaaagctatccatgtaggggctgctccttgcaatgctgcaagcccgctcaatcgcaaacgccccaaacagcagcgtcagggccaaaatacaccagacaatcttattccgcttccatttctggatttcagttgcaatgatcgtcatgcggtttcctcctttcatggttcccattataagggacgaaccttgccataaccttgccgaaatcttgttttaaccttgctttgaacgcaagaaagccctgcgcggtcacagggctttcggaagtatgatggtaaaggttgtaccagctccggggacactggctgccgtgatggttcctttatggacgcttaccagttccttcgcaatggagaggcccagcccgttccctttggcagatcgggagtggtcgcattgatacattcgttcaaagatatgggggatgtcagcggcggaaatccctttcccgttatcggccaccatgatttttgcctgctgctccgtttcggttatggtcagggacacttgccttgcgtcgctgtgcgtcaggatattttgcagcaggttattgagaatacgggtgtaggcggtgggatcaacccttgtcatgtattctgtttcgggtatctctatctcataggaaagattgtgattttccagcagcggcacccagtcagccataatgttgcgggaaagctcgttcaggtcgcagacttcaaaatgaaaaatctgctccccagcatccagcttcacccattcaaacagggcggtcacaaagtctttcaggtgatgggctttttccgtagccacccgaatatattcttcctgctcggcccctgttacgatcttgctttccacggcctccaaatagcccaccagagaggcaagcggcgtttttacatcatgggaaaggcttgtcataagtcgtttataagcctgctcggattgcttttgctggatcagccgggattggctgctcatagcaatttcgttgatgtcataacagatttgctttgtcaggtcactttcccgcgccagcacacggcggttcaggtttccgttctttatatccgtcagggcatctttaataagggaaagctggccccggacgcggtgaagttttgccaaaaggtaagcaatcaccagcagggcaatcaataaggccagcagcagataaatgttcagctccatgctcatgcctccttattgaaccgataccccacaccccggacagtctggatataaaacggctgttctggatttggctcaattttcttccgcagtttgctgataaaagacatgatgttgctgtcgtcaaaggcatattcctccgcccatacctgcgtgtaaatctgcttcttggtaaacacccggcctttgttggacgccagaaacagaagcaggtcaaattccttgccggtcagctctaccgggagattttgaactgtcaccgtccgattgactttatcaatcaccatatctttcagcagcatagtggcggcctcgttcccggccacgggatttaaggtggtatagcgccggatcagggaattgacgcgagccatcagctcgttaatgctgaaaggttttgtcaggtaatcgtccgcccccagccgcaggccagaaactttatcttcctcatcgcttttggcggtcagcatcagcaccggcacattatgtttctcccggatcttctgcaatacctgaaagccgtccatatccggcatcattacatccagaataatcagagaacaggcatccttgttttcttccagcagccgcaggccctctaaaccgccatacgccaccaccgcagacaaattttcctgttccacgcattttttcatcaaggcacaaagttccttgtcatcgtctataatcaaaacactattcatgtttcaaggccctcccttgtttcgtcctgccgtccaccggctttttggcatctttggggattagccaaaccgttcccattttttcagctcccggaatgcgtccctcggagcaataataattgatctgccgtggagaaattccccatttctcgccggcttctcttaaagtcatataatccattgcacatacctccagcacctattatacttcgctctttcgaagaatacaagccaagaaacatgaacgcctaaataagaatatgaaaaataagtataaacgatataaaaggaacctcttgcgttatcaaagcaagaggttcctcatttggtttaatttagccctccaataagagcttcaactttttaaggaccttcattttccgaacatgaacagcattgcggtacacgccaagctcgtctgcatattgccgttctgtcttttcctcaaagaacagcgcctggatcagcgcacgttcctctggagtcagcagggaaagcgcggtatgtagcctgtccacttccagcttgtgaagcaccacgctctccacactttcctgctcgtcggagaattgctgggcattttcatccaccagccggtccaaagaatcttcgcggctggggatgatctgcaccacttggccctcccttccgagaacgaaccgctcggttttcaaatcgttctcaaaatagcgcattttccggtcgccctgcatataggcccggtacacttcatccgtgacctcaacatattggccgcgaatccatattctctttttatcagcgtccacgctgttttacctccgatcagtctgaattgcgagaaattcagactggtcgggcggccccctacaacgtgggaatgatagggttcctctatatagaaaaacgcacctgcagaatgggacaacagacccaacagcaagtgcgtttctttttagttcgtgtaaatcatggcataatagttcagtatgaagggcataatatcccgttgaaaaacgggaagtttttttgaccgtacaccgtcaaacaggcggcctacgacatagaatcatactcgctgcttctaatgagcagcggtgtgcaggatgaccaacgcatgaaatatccctccagacccaaacgtggtctggaggggtgggcatgacaaaccaccctgccaaaacctcgtttttttnattggcagggccagctatacgatcctggtcagttcacttttctccgcactgtcagttctctaaaatttgaactgctatgccattataaaactggcataaatccgtaaaatagaaggagaggtgaactgaaagtgcagaatgaacatgaaacacttggtgagatcatcaagaatgcccgcataaaagcagatattacagtcgaagatctggccgccaaggtcggagtaggcgagagatttatatatcgtatcgagaacgaaggcaaaaagcccagctacgacattctctacaagctgattcgggaactgtccattttacccgatcagattttctttccagaaaagcagattgaggattctgaaatggaaagccttgtccgtatgctgtataactgcgacgagcgttctatgcagatcatcaaggcgacagttagggcggccttggacagccagcccaaagagtaaaaaagagccgatgtccgtgggaaattctcacaggttcatcggctctgcgtctatgcgtctggctctttgatgacaacgatatgtagttgtttcgcctggatcaagctctctcgtttgcacttcgggcagtagagaggataattgattaaaaccgtatcctctctgattcgatcacgggttttattcccgcagacgggacaataaacccactctgtttgctgcatgggaatcacctccgttctcctgttttcaagacatggccgagggcttgctgctttgcaagcccgccggcgtgtctttttnatttattcttccttttcccggaatccccaagcctcaaaatccgtatcgtcctcctccggctccacttcctcggcataatcggaaccggtggcctctaccgggggattgagcatatagggcgtaacctgtaccggcgcttcgctctggatcgccagcaccctacgggcagccttgccgcaaaacaccatggcggcggcaaaggcgaaatactgcacacagcttgtaagataaagattcacaatagccagttcatttccctgcacagggacgccctgctcaagctgccgggagacattttgatggcaggccaccagagagacaaaggctgtaatcagcagcaccaccgcaagagcataaaaaactgcggaaaggatcgttccctttttcgagggtttcgtgattggattgctgtctatctccatcattttgccctcctttcactgcggggagcgttgtttctgcgggattgaggctgggcggctccatcttgaacgcgctctagcacctgcagctcaccctttctcagccggaacaccacatcggcctgcttcgcaagttcattggagtgggtaacgaccaccacacacttgttcatcttgtgggcgctctctttgagaatatccgtgatgtcctgggcggtgtcctcgtccagattgccggtaggctcgtccgccaggatcactggggcctcacttgccagggcacgggcgatggccacccgctgctgttggccgccggagagtttcagcacattccgcttggcttcttctttcgtcagccccaggcgctccagaatgggcagcggcggcagcttggaagtcagcgacacgttctctgcgggggtcaaatagtcgatcaagttgtacgcctggaaaataaacgccacatggtttttccggtggtcggaaagccccgtcttggcaatgtcctgcccatcgaaataaatctggccgctgttagggctgtccaggccgccgatcagggaaagcagggtggtcttgccgcagccggaaggccccaggatggcgtagagtttgccctgttccatttcggcgttgacgccttttaagactttctttttattatcgtaggaatatcccacttgcttgatttccatgatactcatagtgattacctcatttcgttattttaggggggtggatggtcttattccatctgcgacaaaatctgcttgggcttcaatcgcatagtcgagatactggacaccagcaccgccgccaccagcagaatgccgcccacgatagccaccagcatgaaatgctccggggtgacgatgacattctctgccgctttgccgaacagcgtccccagggcgccggcaacctgcttgctggaaaggtaggccaacgggaaggccaccaccgcaatcagcagggtttccagcacatattgtagaatgaccgcaggcttggcaatccccaccgccagcaaaatgccggtttctcgtttccggctgcggatggacatggagaggatgagaattatcaaaaccatgctcaccacagtaatcaccacgatcagcgtcgtaaccaaagtagtcgtatccgcaatagagctggaaatgcgctcgtacacttcattgctcacggtgatgtaataggagttccagtcgatagaggtgctgttctgcacttcctggacaatgctttccagcttagcagcgtccgaaacgaagaagtcaacggaagtaattccatttccattgctggccgtatagggcgcagaaacagccgacatggtatccatatctgtaaaaatatagttctcgtaatcgtataaggttgagggctttgccatagttgcctgctcgtcgtcatcatccgcgatgacctcaaacagaccagcaatctcacactcaaccaacgggtcgtctggattcccgtttttaatttcgagcgtatcgccaagggacaatccatatttctcagccaagccttcgtacaaaataattccactattaccctccgttacatggcagccttctgttaaacgcaaggctccactaagaaacagtggagaatactccgaatcaaagcagcctgttccataaaaaaggtctgctacatattctcgttcatccgtccctttcatcaaagtatcttgattatagagatttacgattgtccaatgggaagtgttataggattctactccgtcaatcgcggcgatttcctcaatcatttcgtctgtgacaggctgcagggtatagccgttgttggcattgacagtaaaactggcccctgttacgccacgcagttcttccgactgctcctcttgtgcgtctgcaatcgccagcccggacattaccagggtggtaatagccaagagcaggaagaacaccagcaaggtctttttccattttctcgcggtatgtaaaaccgctcgtattcctaaattcgttttgtggttcatattgcgcctcctttactccatctgcgctaagatttgcttgggcttcagccgcagggtagagatgctggacaccagcactgccgccaccagcaagatacctcccacaatggccaccaacataaagtgctccggcgcgacgatgacattctctgccgctttgccgaacagcgttcccagggcgccggcaacctgcttgctggagaggtaggccagcgggaaggccaccaccgcgatcagcagggtttccagcacatactggagaatgaccgcaggtttggcaatacccaccgccagtaaaatgccggtttcccgtttccggctgcggatggacatggagaggatcaggataatcagaaccatgctcacagcggttatgacaatgataagggtggtaatcagggtgccggtgtcagaaagggcgctggaaatattctgatatacttcgtcattggcagtaatgataaagttgttccagttaatagaatcaatatgttgcacgttctttattacattgtctaattgagcagcgtcctccacatagaaaaaagctctttcaattccatcatctgagtaatttactgccaatgcctccataacgctgttactacagaaagcatattgattatagtcccaataagtagacgcatcgtacatatttattgtgtcgtccttgtccgccacaatgtcaaatactcctataagctccatctcaacctctggatcgttatactcagggttaattatccctgtgattttgtctcctatttttaatccgtttttttcaacgctttcccggctgagaataattccatctgttacatcctctgtaatatgagaaccttcaacaagctcaaatcgtccagatacaaataaactattaaattcagaattatatgcgccataagcataataggcgaatccgtctgggctaaggctaagattattcccttcttcatcgtacagggtaggcacaccgccatttgacgcatcgtacccagtgataccttctaccgaagcgattttttgaatcatatctttagtgatgagttcttgcgtactataagtaccatcactgtcatttgcccaaccaccagtggataaatttctctctaccgtaaagctggccccggtggttccccggacttcctcagcctgttcctcctgggcgtcggctatggcaagaccggataacaccagggtggtaatggccaggagcaggaagaacaccagcagcgtctttttccacttccgtgcgctatataaaatcgcccgtattcctaaattcatatcgcctgcacctccttaactcatctgcgacaagatactcttgggcttcatcttcatgatcgggtaggccgccagtgcggtagaggccacacacagcactatgccgaaagcccacaccagtgcgtaatcctcggcggaaacaaccacctcaatctcggcaaggcctaaatcctcgttggattcttccgcaccttcgctttctccggtcaagtccaccgtttcataggtttcgcttgtcacctgggagagcaggctgctcccgatctgatgggaaatggctgtgctggttccgaaggagatcaccagggcgatagccgccacaaggatcacttccagcaggtattgcagcagcaccgagcctttggaaatacccatggccagatagacgccggtttcatggattcgcccacggagccgcagcgtcaggaacagggccagtaccaggaaacagatcacggataccactgcaatggcgataaagacgatgttctgcaagccctccaaggattccttggcgttctgatagtcgttgtcatagcgagtcagagtgcagctctcccaatccacaccatctaatttctgcacatcggtcataatgcgctccagctcgtccgggtcgttcacataaaaatccccgtactggacattggtggttccctccgtgccgtagagcaggaaagagacggtggagaggtcggtgaaaacgatgttgtcgtaaaggtcataggacggggccatgccaatggaatcctgttcctcggtgttggtgaagatcccggccaccgtcacggggatggtcttatctttgtcctccacatttcccagcagcatggtgtcgcccacagacaggccattgcgctgggcgaacttctcatggatcagcacctggctgcctgtttcggtggtgatggagttgccctccaccatctcaaagccgccatcggtgaaatagctgtcctcctgggagttggagttggccaccacccgtccagcgttctcatagccctccggcacctgggcagcgccctcggtattgatctccagaaggtttccgtctgcgtcaaaataagtagcgtaggtgtaggagcgcagggtgtacctgccgctcaaaccatccacatcaagaatctggctcaccgtatcctccgggatcccgttttccagcgtcttggcgttgatggtgaaatagcccagcagggcttttttgatattggcgttggctgtttccgtggccgattgaatggaaaggcaggtcagcattaacgtagccatgaccagcagaaacgccagcagcgtgagggtcttgcccttttttcggatggaatacaggcaggcccgtttgcacaggttcatgtctgagccacctcatttccttcgagtaaaaccagtgggttcacttgtgtacctcctaacgtagtttttcgccggggccgtgttaccgccccggtggtgctcccgtctgctgggaacacccttatggtacactacgaaggtggagttgaggtggagttgagatattttttaggtggagttggcgataaggtgtaaaaaaatcggcggccttgatttattcaaagccgccgtatagtcaaacgtaggacgtgacgattacctgctgagcaacggcttttttcttttgccgtctggcggggcagattaaagcgatattcgtttttttacatctcaatcaactcttttgcatttccagtaaatggtacaccataaaantagacctgccaaagtaataattcctttgttccatagccaaatattattttgatctccggcaaatcggataaacaaatagacatattctaaccccggaaatcttaataggcttcgcgtcaataagattatcagccatataaccccacagactaatgttgctatatgttcttgcctacatataatccctaacaaggcacacangsgatrrkaaaaagaatgttggtgggaaatacagcacaatcaattcccatattggcatttcgtatcgcataactgacgcgcctgccatgctaaaaaccgcgaataaactggatattgcaaaagcgattatataacgtaacaatacccatttgaaaagagatttgctttgtgcggcaataatatcaaaattactgtctgctctcgtctttccccattcaccaacaacgattgtgacaaaaaacgggaataccacttcaaagctgaccggataaaaatcaagcagatttccggctatgatagaaagaacaatcatagtaaccgtaaaggccgctgatacgataaagaaatttatcccgaaaatttttacttcgctttcaattcgcgctttcattctgacctccaaaacttgtgtttcaaaaacagcagactgccaaacacaagaccgcttcctacaaggaaaaatccatttgcaataacgctccccatatcaacaaacggccatctcggatgcacccatatcaaacggactaaatcgccggaagtaatcaagtaaaatggcattgcctctgccgagcctacataggtggcattaaacccaaacaatattgcggccagaatatagaataagcgaatgtcaatcatccccggtaaggcaatcgagcaaaagctgacaaataaactcgttggaactaaagaaacaaagattgcttttatcaacgggacaatacattcaatcagggaaatttcaaacggagcagcaataaaatagatcatcgtgttaagcaaaagaaaaatacactgcatggagaaaacatatagaaatccgcccaacagctttcccaacacatattgccattttcgtaaggccgatgacatcagcaaagatttcattcctgtcttagtatctatcggaatacgtcctgccaataagaacatcagaccgaacaataaaacaaggctgtcgaggctcacaatccgataaacaaagtatgctggctcattcaaaaactccaaccgagctaaattcccagcagaggggaaattgtcaagtaaagaaagtgcagtcgctacgagaagtacaccccatgtaaccagacgcttgaactgcattttatattcataatggagaacagaaaacattttacgcatgggctttccctcccatacaatacacataagcatcttccaaagtcgccgtgacaggtgtggcctgtaagaaaagtccgctttcgtccataattcgcgcagtcaatccttggctctcatatgtcgtcgataacactaccgatttcttttgaagctcgataagctggttttcattttctaaatgtaggatttttaccttatcttttacccgttccaataaggtggcagatgtgccagcataaaatagatgccctttccgcagaacagcaagttcttcgcatagctggtgaatatcttcgacaatatgtgtagaaagcaaaacggtgcggccctctcttgcatatcttgacaataggccgcggaacctcacccgttcttctggatcaagaccggctgtcggttcgtcaacaacaagaatttgaggattgccaatcagcgcctgcgcgatgccaaccctccgtttcatgccgcctgaaagctgccctattttgcgtttggaaaattccagcatttcaacttgctccaacacgctactgacagcttttttgttatgaatgcctttgagttctgccatgtaatttacaaagtcaaatacagaaagagaggggtacatggcaagttcctgtggcaaatatccaactcttgctctgacctcatttccctgtttcacggtatcaaaaccgcaaatgcggattgtgcctgctgatggggttaataccgtaaccaatgccttcatcagcgtagtttttccggcaccattgtgtcccaacaggccaaaaacaccgctgccaacatgaaagctgacatcatcaagggcggtcacgccgctcttgtattttttagtaatgtgactaatctcaatgtttgtattcatacgctcctccatttaactacaatcgtagatttatagactttaatgatacgggttactcattgtaacccgcaccatattctgtaccagtatgataggccggataaggatagaaatgttctctgctgaattgtacggaaaaagcaaccgtaagtatcagagaaacagccagaatagccgttatcatcctgtccctgatctttgatgattttgtggtgatccgtttgattctacgttttgtcaacacaaaactgagtgcgccaatccctttaccgggttttgtagcttgaaccgtagacagttccagcagtcggataatagccgcagcatattctgtttctgtgacttttccctgcgaaaattttaaggcattgcaatcgcaatccgtttcacaatgtagtgaaaaatctcctttagcaatccacgcaaaagggttgagccagtttaatatgcaaacataatcatagatacgctccaaaagaatatgtttccgcttaatatgggtcaactcatggataaagacagcggataattcgccatcggtcagcttttctataactgttttgctcatgataattgctgggcggattgcgccggtaacgcaaatggggttatccaaagtcccccaatatagtgcaggcactttttttaagccgacaatttccgcacattgtaagcaaagagcatttagacgctcagtgctacatatctggtatcctttcaatgcccgctttgccattatagagcgtatcgtatcatagacgaaaagaataccgcacccacacacccatacccaacaggcaatataaaatccatttaaccattcctgtgtttctggtgatatgagaaacacatccggtgaaaggaatggaacgcaaaaaaggatcataccgcaaagcggaaaccatagcggcagcgtacaacgcagaatgtgattgataaaaagagataaaacacaaaatataccgccaatagcagatacaaaaataatcgcattgaaaatgttaccgagtatctgcatctgttttctcctgctgctcgatcaagctctttagctcatgcaattcatcagacgaaagattgccgtttttgacaaatcccattaccatcttagacagggaaccatcatatacgcggcttagaaaactgcggctttccgcaagctggcattcactttctgatactgctggataatataagtatccctttccttgacgttttgtagtcaatgctcctttttttaccaaacgggcaaggtatgtttgaatagtcgtcttgctccaacctgtttcttttaacctgtctaatatttcgggaagtgtttgtggagaattactccaaacaatcttcattactcgccactctgactctgaaatagaaagttgctgctccataaagcgcctcctttgattgatgctgtgtagtttattctattatagcattggaactacatagtgtcaaccattgcatagaatatctatacgtaaaattaaacggaaaactatgcctgtctacgcatttaagccactgaaaagcccgctatttcggggtttcccggcatggtcgatgggcaaggcaggacagattatctaatcctcgccagagagcgcttctaaatctgtagggaaatccataaaacgttgggcatggtaactgttagctacacacccaacgctttttttcataagagtttatagtgccatcaccacaaaaatgctgtaaacttcacaccgttttccgtattaaccatgttatagataatttcgtggtgatccaggatggttttagtgatatacagccccaggccgctgccgccgctgttccggttccgggatttgtccacccgatagaagggagtgaagatacgctccaaatcttcctcagcaatgtggacgccggtattctccacggaaaacacctcgtctttttgggtgacagtaatcaccgcccccaccggcgaataggtcacggcatttccgatcacattggtgaacactttctccataagccgacgatccccctggtagtggtatcccggctggatgtccatgcgaagctcgatctgcttgtcctccattcggccacggaactgttggcagacctttcgcaacatttggctgatgtccaggtcggtacgctccaggtggaaatcgctgccgcccatccgagccgcagaaaggatttctttcaccaatgcctccatgtcatttgtggttttcagacaccggcgcaggtaagtatcgcggtctttgtattcgcccacctgatagatcatgccctccagttgccccttgatgatggcgatgggagttttcagctcatgggatacagcggtgaaaaattccacgcgctgcttttcctgttcccgttctcgctcaatatctttcttcaactgccagtttgccgagcgcaagctggccagcgcattgttgagctgctccgccatttcgttcaggctggcggccagcacaccgatctcgtcttgccggttgacttcacacttccaggtcatgtccagggtggtcatccgctttgccacattgctgatactgaccagcggcttagagtagtaccgggaacaaactaccgccccaatgacggagatcaatagaatcacaacggcaataaaggggatgagcttcaacaggatgtcataggactgcgccaccgcaaccagggcggcattggcaaatacatggtaggtctgcccgccttgctggaatgtagcggaacagctcatagagggggcagaagtgtccatgttctccatatcggcaaaattcacggagaacaaattattcccataatcatcgttgatttccaccgaggcgttgttcgtcatggaaaattccatcagactgtccgaactggcctcccagccgtttcgctccaaaaccgccaccaagtcataaaaatcggaagtgacttggccttccagctccgtatggtaattcctcggcaggaaaatcatcaccatgccgtaaatgataatgcagcagaccaccagcagcgccagcatactgaggaaggttttggcctgaatgctttgactaatcttccttctcaattttatatcccgcccctctcatggtttctatgtagtccacacccagctttttacggatgttcttgatgtgggtattcacgattttttcatcgccaatatagtcgtagccccaaaccgtgtccagcagcctttccctggacaccacccggccctggttctccagaaggagcttcaaaatctcaaattccctggtggtcagggaaacgctttctgttccaacaaggacggtaaggctgtccgtgtccaccgtgatttccttatagcggatcacggtattgggcgcagcgccgccctgttcagcccgacgcagcaccgcttcgatatgcttcatcacaaccggcatggaaaagggcttggtgatataatcatctgccagagcgtcaaagcccttcatctggctatcctcatcatccaatgcggtgagcatgatgatgggaacccggctctccttccgcagaatctcacatacggcaaagccatcaattttcgggagcattaaatccagaagcaccaggtcgggcttgtttacccggaactgttcaattccttccaggccatcagcagccagaaccaccgtatagcctgcgtcctccaggaacgccttgataatattctgaatggattcttcatcctctataatcaaaatcatttttgacatcgcttgcacctccaaaggtagtgtaacatacaaaggtggagtttaggtggagaagggggccttttttgaaaaacctctctattcatattatacctgaactgtgttgccagaaaaagtgtgcgagtttgaattttgtatatcctcatagcacgagattgtatgacaggctgtttctatttgagaaagtcaaaaatgcccctcacccaacaatatccaggcgaggggcattagatttaactctccaataagagcttcaactttttgaggaccttcattttccgaacgtgaacagcattacgaaacacgccaagctcgtctgcatattgccgttctgtcttttcctcaaagaacagtgcctggatcagcgcacgttcctctggagtcagcagggaaagcgcggtatgtagcctgtccacttccagcttgtgaagcaccacgctctccacactttcctgctcatcggagaattgctgggcattttcatccaccagccggtccaaagaatcttcgcggctggggatgatctgcaccacttggccctcctttccgagaacgaatcgctcggttttcaaatcgttctcaaaatagcgcattttccggtcgccctgcatataggcccggtacacttcatccgtgacctcgacaaattggccgcgaatccatattctttttttattagcgtccacgctgttttacctccgatcagtctgaattgcgagaaattcagactggtcaggcggccccctacaacgtggaaatgatggggttcctctatatagaaaaacgcacctgcagaatgagacaacagccccaacagcaagtgcgtttctttttagttcgtgtaaatcatggcatgatagttcagtgtgaagggcgtaaagccacgatgaacagcaagcggcttttttgatagcctgcgccctgtcgcattatgtcgaaacgctctttgtttcatggcggctccctcctaaagccgccgtgtcagcgtataggcgtcactcctttgttgagggcataaggaacggcggccttgatgttactcaaaaccgccgcagtaccctgaaaatccattatgggcgcacaaagctacctgccctgcaacggttttttctttccccgtttagcggggcaggatagcttgtcttattcgttttattcttgctcttgtgcggctgcctctttcagccgcgaaaagctctcatcgctgataatgtgttcaatccggcaagcgtcggcctccgcagtcctggggtcaacgcctgcggcgataagctgctcggtaaaaaagcaatggcgctcatagattttttcagcaacctcgcggcccacatcggtcagatggagaaagtgatcttcgtccattgtaagaaagccgccgtcccgcaaggtagccactgcatggcacacgctgggctttgacacctccatgtgccgggccacatctacggagcgcaccataccgagtttcttttggagaacaaggatggtttccaggtagtcctccccggacggagttattacgggcttcataaactctttgcgctcctttgcctgtttttgttgcaattcttcccatgcttcacgaataatcaaagaaaacaccttggcacgccccctttcaagaataataggttctgatgtttatgcaaaagaaatcactttagtttccagccacttgccagttcacgctgtttccaaaaacgactataaattccatcttgagaaatcagttctgcgtgtgttcctttttgcgccactttgccatccgatataacgacaatctgattggcattttttactgttgataagcgatgtgcaatggaaatcagtgttttnccctttgtaagctgttcaatcgctgccaacagttcatgttcattctccgggtcaacgctggatgttgcttcatccaggataatgattggagcatctttcaaaatggctctcgctatggaaatacgctgcttttcaccgccagacaaagtagagccaccttcacccacctttgtgttatatccttcgggcaaagatacgataaagtcatggcattgagccttttttgctgcctccacgatttgctcatggcttgcatcaggattgccaaagcgaatattattctcaatggtatcattgaaaaggtagacgctttgaaagaccatgctgatatattgcataaggctatccgcagtgtaattttttacatcctgcccgtccacaagagcctgaccttcttgaacatcccaaaagcgggcaatcaggttgcaaagggttgttttacctgatcccgatggccccacaatcgcgcaggtggttccctgtgggataagcaaagaaacatggtcgataatttttctggacttttcatatccgaaagatacatcccgaagctcaatatcaaaccgtttcgggacaagtttatttgatgtcgtgtccatctgcgggatgttagtaacttcttccagacggtttaagccagcggtgacgacacgggaaagaaacgcaccatttcccatctgctccatttcggaaaaaatgataaaggcactgacaatcaccatgaggcaccacggcaacgataaacttcccatcagatacaaaagaacggcggctaacatcaaaacactgccggttatcttgtaaacagcagagtaccatttcatcaatgccatagaagcgtgttcctgtgcaagatcagcttctttctttttgcgaaaagaggcataaacggcattttcgctaccatccgcttgggaaaaggcacgaagcaccgcaatcccccggatatattccaacgcctgagtcgtcatatcttcttgcgccgccaatactttcggcgcgtgttcccttgcaacttgtccaatagcatgaagaattaggatgccgacaagggaacctgcaagggtaattactgcaaaaacaggaatggaaaataggaagaaaacgatcattaccagcgccatcaacgctccgctggtcagatcagtgaccgccatcatggaatattgttccaactctgttactgtcgttgtaagtacattctgaatacttcccaaacgctgctcggaaaaatatcccataggtgcttttcgcatgagggcgccaacatgaagtctgtaatcacggaatatatcaaagcccttagcactcatggaaatatccataagccactgaaaaaagaaacggcccaacacactgatacacaagacagcaagagctttccagatcacatccattgtcaggctgtccagatcagaggcaacaatataaaccgcccataaaaccattgccata

SEQ ID NO: 6 - DNA, 55 bp, artificial
  A1EcoRfw
ttttgaattctttaggaggttttttatgtttcagttaaagtgggtgtggaagcag

SEQ ID NO: 7 - DNA, 51 bp, artificial
  A2XbArv
tttttctagatcagctctccagctgggccgtgtacagatggtaataatatc

SEQ ID NO: 8 - DNA, 3537 bp, Unknown
  misc_feature: Gram-positive bacterium from the phylum Firmicutes
atgtttcagttaaagtgggtgtggaagcagatggagggcttccgaaagcggtacatctttgccctgttctccacggccctgctggcgatgctgaccctgggcaactcagtcatcaccgccagcattatggacacggtgttccagccgcttaccgaaagcggtgtggtcacccagcaggtggtgcaccatctggcggttctggttgcggtgctcattgggtttacgttgttccgcaccagctttcagtacctatccattatgacctatgagggctgttcccaaaagctaatcttcaagctccgccgggatttgtacaagaatatgcaggagcaggaccaggacttcttctccaaaacccgcaccggcgacctgatgacccgcctcaccggcgacctggatatggtccggtttgccgtggcctgggtggtgcggcagctgatccactgcacagtgctgtttgtcaccacctccattgtgtttttggtgacagactggctctttgccctgtccatgctggcggtaacgcccattatctttgccctgacgctggccttctccaagcgggtgcaccccctgtatgtggacctgcgggagcgcctctctttgctaaacacccaggcccaggagaacatctctggcaaccgggtggtaaaggccttcgcccgggaggactatgagatcgaccggttcgatgagaaaaatgccgactataaaaaggccaacaccagggcctccctgctgtggctgcagtacagcccctatattgagggcctgtcccagtccctctccattgcggtgctgctggtggggggcgtgttcctcatcaccgggcgcatctcaattgggaccttcaccctgttcaacggcttgacctggacgctgaccgaccccatgcgcatgttgggcatgcacctcaacgacctgcagcgtttcttcgccagttccaataagattatcgagctgtactatgccaagtccaccatcacttcccggcccgacgccaaaaaggtggacacccgcctgaagggggagatcgacttctccggcgtggacctgaacctccacggccaaccggtgctgcgccacattgacctgcatatcaatcctggcgaaacggtggccattatggggcccaccggctctggaaagacctcgctggtgaatttgatcccccggtttacggacgtcagcggcggcaccctcaccatagacggcacgccggtggggcgctatgacctgcagggcctgcgccacgctatcggtatcgccacccaggacgtgttcctgttttccgacacggtggacggcaatatcgcctatggcgactcctccctctccgaggatgacgtgaagcgctatgccaaaatggcggatgtggactttgtggaaaagcttcccgatggcttcgacaccctcattggcgagcggggcaccggcctttccggcggccagaagcagcgcatcgccctggcccgggccctggccgtgcggccctccatcctcatcttggatgacaccaccagcgccgtggacctggaaaccgagaagtacatccaagagcagctggccagcttggacttcccctgcaccaagatcatcgtggcccagcgcatttccaccaccaagcgggcggataagatcgtcattttggataagggccgggtagtggacatcggcacccacgaggagctgtcccagcggcccggctactaccgggaagtgttcctgctgcaaaacggcatggaagaggaaaaggaggtggtttaaatggcacgcaacaagtttgacgtggacgaaaccctggaaaccccatttaacataaagcatctgctccgggccggcgtgtacattggccgccacaagaaaaagatgattttgtccctgctgttctccgccatttccgccgcctgctctctgctggggcccatgctcatccagcgggccattgacgtgtccgtgccccaaaaggactacaccgagctggtggtgctggccgtcatcatgctggtgtccattgtggcctctgtgctgttcgcccgggcccgctccaagtacatgattgtggtgggccaggagattatctacgacatccgcaaggacttgttcgagcacctgcagaagctgcccttccagttttacgatgaccggccccacggcaagattttgacccgcgttatcaactatgtcaactccgtgtcggatgccctctccaacggcatcatcaactttgtgctggagatcttcaacctgatcttgattgccgtgttcatgttcctgtgcgatgtgcgcctgagcctgatcgtcatggcgggcatccccctgtttctggtcatcgtgctgctcatcaagcccgcccagcgccgggcctggcaggatgtgtccaacaagagctccaatatcaacgcctacctccacgaaagcctggacggcatgaagatcacccaggccttcacccgggaggaggagaaccgcggcatctacgagaagctgaacaagaagtgctatcagacctggatgaaggctcagtacacctccaacctggtgtggtactctgtggacaacatctccacctgggtggtgggcgccatgtacctcatcggcctgtggatgctggggcccgccatgcagattggcaccctcattgccatttcctcctacgcttggcggttctggcagcccattttgaacctgtccaacctgtacaacaccttcatcaacgcggtggcctatctggagcgcatcttcgagatgattgacgagcccgttactgtggacgatgcccccggcgccaccgagctgccccccatcaccggccgtgtcaccttcgatgacgtgaccttctcctatgatgggcaaatcaacatcttagagcacttcaacctggatgtaaagcccggcgagtccattgccctggtgggccccaccggcgccggcaagaccactgtagtgaacttgatctcccggttctataacatcgacaggggacgcctgctgctggatggccacgacatcgcccaggtgaccctccgctcccttcgctctcaaatgggcattatgcttcaggacagcttcatcttctccggcaccattatggacaacatccgctatggccgcctggacgccaccgatgaggaggtcatcgccgcggccaagaccgtgcgcgcggatgagttcatccgggaaatggaggatggctattacacccaggtcaacgagcggggctcccgcctgtcccagggccagcggcagctggtggcctttgcccgcaccctgctctccgaccccaagattctggtgctggatgaggccacctcttccatcgacgccaaaaccgagcgcctggtgcaagagggcctcaacgcccttctgaaaggccgtaccagctttatcatcgcccaccgcctttccaccatcaagaactgcgaccgcatcctgtacatttccaacaagggcattgcggagatgggcacccatcagcagctgttggagaagaagggatattattaccatctgtacacggcccagctggagagctga

SEQ ID NO: 9 - protein, 1177 aa, unknown
  MISC_FEATURE: Gram-positive bacterium from the phylum Firmicutes
MFQLKWVWKQMEGFRKRYIFALFSTALLAMLTLGNSVITASIMDTVFQPLTESGVVTQQVVHHLAVLVAVLIGFTLFRTSFQYLSIMTYEGCSQKLIFKLRRDLYKNMQEQDQDFFSKTRTGDLMTRLTGDLDMVRFAVAWVVRQLIHCTVLFVTTSIVFLVTDWLFALSMLAVTPIIFALTLAFSKRVHPLYVDLRERLSLLNTQAQENISGNRVVKAFAREDYEIDRFDEKNADYKKANTRASLLWLQYSPYIEGLSQSLSIAVLLVGGVFLITGRISIGTFTLFNGLTWTLTDPMRMLGMHLNDLQRFFASSNKIIELYYAKSTITSRPDAKKVDTRLKGEIDFSGVDLNLHGQPVLRHIDLHINPGETVAIMGPTGSGKTSLVNLIPRFTDVSGGTLTIDGTPVGRYDLQGLRHAIGIATQDVFLFSDTVDGNIAYGDSSLSEDDVKRYAKMADVDFVEKLPDGFDTLIGERGTGLSGGQKQRIALARALAVRPSILILDDTTSAVDLETEKYIQEQLASLDFPCTKIIVAQRISTTKRADKIVILDKGRVVDIGTHEELSQRPGYYREVFLLQNGMEEEKEVVMARNKFDVDETLETPFNIKHLLRAGVYIGRHKKKMILSLLFSAISAACSLLGPMLIQRAIDVSVPQKDYTELVVLAVIMLVSIVASVLFARARSKYMIVVGQEIIYDIRKDLFEHLQKLPFQFYDDRPHGKILTRVINYVNSVSDALSNGIINFVLEIFNLILIAVFMFLCDVRLSLIVMAGIPLFLVIVLLIKPAQRRAWQDVSNKSSNINAYLHESLDGMKITQAFTREEENRGIYEKLNKKCYQTWMKAQYTSNLVWYSVDNISTWVVGAMYLIGLWMLGPAMQIGTLIAISSYAWRFWQPILNLSNLYNTFINAVAYLERIFEMIDEPVTVDDAPGATELPPITGRVTFDDVTFSYDGQINILEHFNLDVKPGESIALVGPTGAGKTTVVNLISRFYNIDRGRLLLDGHDIAQVTLRSLRSQMGIMLQDSFIFSGTIMDNIRYGRLDATDEEVIAAAKTVRADEFIREMEDGYYTQVNERGSRLSQGQRQLVAFARTLLSDPKILVLDEATSSIDAKTERLVQEGLNALLKGRTSFIIAHRLSTIKNCDRILYISNKGIAEMGTHQQLLEKKGYYYHLYTAQLES